(19) United States
(12) Patent Application Publication
(10) Pub. No.: US 2019/0290287 A1
(43) Pub. Date: Sep. 26, 2019

(54) Compressive Brace for Intraarticular Needle Procedures

(71) Applicant: Biosurgery Solutions LLC, West Orange, NJ (US)

(72) Inventors: Wilmer L Sibbitt, Jr., Albuquerque, NM (US); Philip Arthur Band, West Orange, NJ (US); Randy Robert Sibbitt, Helena, MT (US)

(21) Appl. No.: 16/316,705

(22) PCT Filed: Jul. 3, 2017
(86) PCT No.: PCT/US17/40617

§ 371 Date: Jan. 10, 2019

Related U.S. Application Data
(60) Provisional application No. 62/361,857, filed on Jul. 13, 2016.

(51) Int. Cl. A61B 17/132; A61F 13/06; A61M 27/00; A61F 13/00; A61F 13/10

(52) U.S. Cl. CPC A61B 17/1325; A61F 13/062; A61M 27/00; A61B 2017/3413; A61F 13/066; A61F 13/108; A61F 13/00017

(57) ABSTRACT

A compressive brace for intraarticular needle procedures that provides constant radial compression. One embodiment comprises a non-pneumatic releasable compressive planar device or tubular sleeve, a method to apply tension to the device or sleeve comprising a non-stretchable material or stretchable elastomeric material with fasteners or external compression straps or both resulting in fluid displacement and increased intraarticular fluid pressure, a design that displaces fluid within the joint to low pressure areas to be readily accessible by needle or catheter via specific anatomic portals so that the joint can be completely aspirated, tension and pressure can be released so that fluid can be easily removed or injected into the joint, and integration of the device into arthrocentesis, surgical, diagnostic, and joint therapy kits. Some embodiments define a window or aperture to allow pooling of fluid and to introduce needles and catheters. Some embodiments dilate the patellofemoral joint and may displace the patella-making introduction of the needle more successful.

### TECHNICAL FIELD

The present invention relates to an orthopedic appliance for facilitating, diagnostic arthrocentesis, therapeutic arthrocentesis, injecting medications into the knee, diagnostic imaging, image-guided procedures, and joint therapy in the knee or any other joint.

### BACKGROUND ART

The knee is the most common joint that is aspirated in a procedure called arthrocentesis and injected in a procedure called intraarticular injection. The fluid that is aspirated, called synovial fluid, is used to diagnose infections, inflammatory or crystal arthritis, and other diseases of the joint, and in the knee 2 ml is considered a successful arthrocentesis with enough fluid for cell counts, examination for crystals, gram stain, and culture and antibiotic sensitivity. The knee joint is also commonly aspirated for purposes of administering pharmaceutical agents into the knee joint such as glucocorticoids or visco-supplements (hyaluronate derivatives) in the procedure of intraarticular injection. A medical practitioner must have basic skills and knowledge in performing arthrocentesis and intraarticular injections of the knee and other joints, and typical medical specialists who perform these procedures include orthopedic surgeons, rheumatologists, emergency medicine specialists, sports medicine physicians, or a general doctor amongst others. Even with experienced specialists, knee aspirations or injections can be difficult to perform because there is a very small area in the synovial membrane space in which the aspirating or injecting needle can be inserted in the joint without striking pain-sensitive structures including bone or ligaments or missing the intraarticular space which is quite small and generally less than 1 mm in width, thus, is potentially difficult space to access with a needle. Further, the fluid coats all internal surfaces of the joint and thus is not concentrated, but rather distributed throughout the joint, synovial reflections, bursae, and joint surfaces.

Nevertheless, complete arthrocentesis (that is, completely aspirating all fluid from the joint) before injection of a medication has been shown to improve the outcome of injected hyaluronate and corticosteroid, emphasizing the need for complete arthrocentesis of a joint before injection of an intraarticular therapy (Weitoft T 2000, Tanaka 2002, Zhang Q 2015). Indeed, for commercially available hyaluronates, the package inserts typically recommend complete arthrocentesis before injection.

Thus, there is a need for new methods to permit more successful arthrocentesis for diagnosis, more complete arthrocentesis, and more accurate needle placement to improve outcomes of intraarticular injection therapy.

There are a number of orthopedic appliances that are used to support or immobilize various body parts. Compression braces or sleeves are often used after or during procedures to prevent swelling, fluid accumulation, or deep venous thrombosis (Kuster M S et al 1999, Westrich G H 1998). Static or non-pneumatic compressive devices made of virtually any sturdy fabric but more commonly are made of stretchable/contractile elastomeric fabrics such as neoprene, fabric-combined with neoprene, or other plastic or rubber like fabrics (Lawrence D 1980), however, mechanical and pneumatic devices combined with fabrics are also used (Janssen H et al 1993, Westrich G H 1998).

With respect to stabilizing or immobilizing the knee joint because of an injury, certain of these orthopedic appliances include inflatable air bladders for intermittently supporting and/or releasing support on the knee. One example of an inflatable knee brace includes U.S. Pat. No. 3,983,056, which describes inflatable tubes stitched into a fabric support extending vertically over a portion of the support U.S. Pat. No. 4,430,042 describes a pillow type device strapped to the leg of a patient and then inflated. U.S. Pat. No. 4,872,448 discloses a U-shaped inflatable bladder placed over the patella. U.S. Pat. No. 4,938,207 describes a linear brace employing first and second fluid filled chambers. U.S. Pat. No. 4,947,834 describes a brace for compressing a patient's outer extremities, the brace including flexible chambers arranged in a series that are then successively inflated. U.S. Pat. No. 4,960,115 describes a body support apparatus having at least two inflation chambers. U.S. Pat. No. 5,626,557 discloses a knee brace having inflatable supports extending longitudinally on both sides of the knee.

U.S. Pat. No. 7,468,048 to Meehan 2008 discloses a compressive device for joint aspiration whereby the pressure is provided by integrated pneumatic bladders that fill reversibly with air to provide pressure much like a blood pressure cuff to move fluid to a predetermined aperture (window) that is not compressed by air bladders. When this device is placed, air pressure within the bladders provides and determines compressive force rather than manually applied compressive straps and or elastomeric and recoil properties of the brace itself, thus, the function of this pneumatic device is entirely dependent on air pressure and integrity of the air bladders rather than intrinsic compressive force of the brace itself. A problem with this pneumatic device is that it is complex, if disposable is expensive due to the airbladders, or if it is reusable, is difficult to clean or remove the bladders into a new sterile sleeve and difficult to clean or protect the sleeve. Unlike a non-pneumatic brace, a pump or other ancillary device is needed to apply pressure to the air bladders and a valve required to release pressure. All of these components result in complexities and increased cost. Another problem with this device is that needles can puncture pneumatic bladders, and an errant placement of the needle would damage the bladder and make the device unusable.

Also, in the pneumatic device as described in U.S. Pat. No. 7,468,048 to Meehan 2008 the size or diameter of the portal was not described or anticipated as crucial, as portal size or diameter is crucial in determining whether localized pooling of fluid can occur as we describe fully in the present invention. Another problem with this device, is that there is no intrinsic limitation other than rupture of the pneumatic bladders to control the compressive force, thus, just like a blood pressure cuff, with a pneumatic device it is possible to provide so much pressure to the extremity that the arterial blood supply is shut off, causing ischemic and compressive damage to extremity creating a safety issue. Unlike a blood pressure cuff that is immediately released for the blood pressure reading, if this pneumatic compression brace is overinflated and is left in place for the duration of the procedure that could take 5 to 15 minutes depending the complexity, decreased blood flow to the extremity could result in a serious crush and ischemic injury.

### SUMMARY OF INVENTION

Positioning of the knee and the particular needle approach are important for arthrocentesis and intraarticular injection success as with knee extension the fluid pools in the lateral recess of the suprapatellar bursa (Hirsch G 2012), thus, in palpation-guided anatomic landmark arthrocentesis or intraarticular injection the extended knee provides fewer dry taps and more fluid and more accurate needle placement (Zhang Q 2012). Because of these considerations, the extended knee lateral superior approach is generally recommended for arthrocentesis (Roberts W N 1996) and the extended knee lateral midpatellar or lateral superior approach is recommended for injection of medication (Jackson D W 2002). U.S. Pat. No. 5,634,904 to Battenfield describes a knee injection sheet that has predetermined portals to assist in needle placement, but does not provide the knee with focused compression to move synovial fluid from high pressure areas to pre-determined sites of low pressure were fluid can pool and distend the synovial membrane to improve arthrocentesis and intraarticular injections, moreover, the individual differences in body dimensions between individuals preclude the universal application of this type of device as the positioning of the portals will be different for different sized people. Thus, methods and devices that would force synovial fluid from difficult to access areas to predetermined low-pressure areas would improve arthrocentesis success and yield.

Importantly, there are clinical reasons why alternative anatomical approaches to the lateral suprapatellar extended knee/supine approach for arthrocentesis may be valuable in certain clinical situations. Further, certain practitioners prefer the medial approaches even though existing data suggest these are less successful approaches (Roberts W N 1996, Jackson D W 2002). In certain situations, the flexed knee (bent knee) position, such as a debilitated person in a wheelchair who cannot lay supine, a person with knee contractures, a person with abdominal pain who cannot extend their hip and knee, or a person with spinal stenosis in whom sitting with a flexed knee is a preferred position (Chavez-Chiang C E 2011). A method and device to increase pooling of synovial fluid in the target of the bent knee position (either lateral suprapatellar bursa or synovial reflections of the cruciate ligaments or femoral condyles) would be valuable in these situations.

Contraction of the quadriceps muscle increases intraarticular pressure and has also been noted to cause fluid to move to the lateral aspect of the suprapatellar bursa and is useful to detect small effusions, and these data provide a rationale for other methods to increase intraarticular pressure other than muscle contraction (Ike et al 2010). Unfortunately, muscle contraction increases the resistance of the tissues transversed by the needle and is more likely to cause bleeding, pain, and damage to the needle or catheter. Thus, alternative external methods that permit the patient to relax their muscles while increasing intraarticular pressure and fluid pooling for arthrocentesis and joint injections would be valuable.

For very large effusions, vacuum arthrocentesis has been recommended (Nahir 1984). Vacuum arthrocentesis increases the pressure differential from the intraarticular space to the fluid collection container (vacuum bottle or vacuum syringe) permitting more rapid arthrocentesis. However, such devices can be awkward and do not change the location of pooling of intraarticular fluid within the joint. Further, the extreme vacuum of vacuum bottles may cause the synovial membrane to collapse around the catheter orifice, impeding fluid movement. In the present invention we demonstrate that a pressure differential to assist in arthrocentesis can be created by external compression yet also change the anatomic pooling of fluid within the joint to a predetermined portal or access point.

Ultrasound has permitted greater accuracy and fluid aspiration success than traditional palpation guided anatomic methods (Wu T et al 2015, Sibbitt 2012, Wiler J L 2010). However, ultrasound guided arthrocentesis requires training, the equipment is costly, the procedure is time-consuming, and the procedure is expensive with many insurance carriers and health plans refusing to reimburse for ultrasound guided knee procedures, despite the improved outcomes (Sibbitt 2012). Further, ultrasound permits joint fluid to be detected but does not move fluid to where it is more accessible. Also, ultrasound may not detect small amounts of fluid that are layered over the synovial membrane and cartilage surfaces. Thus, there is a need for low cost, non-imaging methods to improve arthrocentesis success and yield and intraarticular needle placement for injection that provide similar or superior outcome as ultrasound guided procedures at less cost and can provide small amounts of fluid even when not visible by ultrasound.

Local compression with the hands to assist in arthrocentesis is often recommended and used by physicians, but there are no systemic studies to demonstrate greater effectiveness of compression with the hands (manual compression) (Zuber T J 2002). First, it is difficult to place constant pressure with the hands, as the tension naturally varies with muscle contraction in the hands, the operator eventually tires, and even two hands cannot compress the entire joint 360 degree surface surrounding the joint. Also, a single operator has difficulty compressing the joint with one hand while aspirating or injecting, and a second operator to compress is better, but there are expenses associated with a second operator. Further, when the hands are placed within the operative field there is markedly increased chance of needle-stick and acquiring blood borne diseases, thus, compression with the hands in the operative field is dangerous and is to be discouraged in needle procedures. Finally, synovial fluid is very viscous, being almost a gel, and moves only slowly in the joint with pressure—thus, variable pressure as done manually will cause the fluid to vacillate, rather than move predictably and fully into low-pressure areas. Thus, there is a need to provide effective local constant compression without using human hands to compress the joint.

Synovial fluid from the joint is typically analyzed for volume, cell count, cell differential (red blood cells, neutrophils, monocytes, etc.), the presence of crystals, the presence of bacteria or fungi, and in more recent times, for biomarkers to determine arthritis activity or etiology. The standard is presently 2 ml to obtain these studies. Thus, a method to permit enhanced success and volume in sampling of synovial fluid for analysis for all these purposes would be extremely useful.

Embodiments of the device as in U.S. Pat. No. 7,468,048 to Meehan 2008 without windows or apertures were not described or anticipated, nor were embodiments for the bent or flexed knee. A portal or aperture makes contamination with blood or leaked synovial fluid of the compressive device more likely, and conversely, makes contamination of the puncture site more likely, thus, a pneumatic and/or pneumatic brace without windows or apertures would be useful. Further, a unitary brace without intrinsic windows that creates a low-pressure access area created by the boundaries of the straps and the margins of the brace body was not described. In addition, although the application of the pneumatic device with portals for arthrocentesis was described, the specific use, method, application, and design modifications of a compressive device to assist in joint procedures other than arthrocentesis such as joint injection with corticosteroid, hyaluronate, or other medication were not described or anticipated. Finally, the use of a compression brace to decrease procedural pain was not described in U.S. Pat. No. 7,468,048 to Meehan 2008.

As an example, a pneumatic blood pressure cuff is used to measure blood pressure, but a pneumatic cuff is not used to dilate veins and draw blood or inject medications into a vein with a needle for all the negative reasons above, rather an elastomeric tourniquet is used instead. For all the same reasons that an elastomeric tourniquet is used to draw blood rather than a pneumatic cuff, a non-pneumatic device is preferable for needle procedures of the joint as we elaborate in this invention, but was not described or anticipated in U.S. Pat. No. 7,468,048 to Meehan 2008.

Thus, there is a need for a simpler, less expensive, and safer non-pneumatic device that does not contain inflatable bladders, can be disposable or durable and washable, does not require pressure sources and valves, is constructed of such a material or fabric so that complete occlusion of the arterial supply is difficult and unlikely and is thus safer, can still function after a needle puncture, may decrease procedural pain, can be used on both the extended and flexed knee, may have portals of a specified size or diameter to permit actual pooling, and is easily releasable that performs some of the similar functions as the proposed pneumatic device of U.S. Pat. No. 7,468,048, but generates pressure by the non-pneumatic binding nature of straps and/or elastomeric contraction that can be used for arthrocentesis, and intraarticular joint therapy and injections. Further, a pneumatic and/or non-pneumatic device designed specifically for the bent (flexed knee), as well as a pneumatic and non-pneumatic device without portals or apertures would be useful.

In the prior art, there are described windows in the compression sleeve, most commonly to accommodate the patella (knee cap) or prevent pinching in the popliteal areas as in U.S. Pat. No. 5,139,477 to Peters. However, these windows do not permit pooling of fluid and are smaller in diameter than the patella. U.S. Pat. No. 7,468,048 to Meehan 2008 discloses a pneumatic compressive device for joint aspiration whereby the pressure is provided by integrated pneumatic bladders that fill reversibly with air and access openings permit access for arthrocentesis. A problem with these described access openings with the pneumatic compression sleeve are that the approach to the joint is defined by the location of the access opening while actual practitioners may use a number of different anatomical approaches to the knee. Further, a window placed strategically and large enough to permit the fluid to pool beneath the patella was not described. Moreover, windows or access apertures in an elastic, plastic or cloth non-pneumatic compression sleeve to permit areas of low pressure, pooling of synovial fluid in those areas of low pressure, adjustable placement of these windows by different placement of the device, and multiple portals of access to that fluid have not been described, and are described in the present invention.

An additional problem is that there has been no description as to the size or diameter for access apertures necessary to permit access to the pooled fluid, yet not compress the overlying tissues. Thus, the aperture has to be of sufficient diameter to permit the underlying tissues to stretch without overlying tension to accommodate anatomic synovial fluid pooling otherwise if the aperture is too small, the tension is transmitted to the knee tissue and no pooling occurs. Thus, there has been not been a description of the dimensions of the windows or access apertures in an elastic, plastic or cloth non-pneumatic or pneumatic compression sleeve to permit areas of low pressure, pooling of synovial fluid in those areas of low pressure, and multiple specific anatomic portals of access to that fluid have not been described, and are described in the present invention.

The prior art, principally U.S. Pat. No. 7,468,048 to Meehan 2008, does not describe a brace that can be used in the bent (flexed knee). Rather, a pneumatic brace as described in the prior art would force the leg into extension with the bladders. Thus, there is a need for a device that can be fitted onto the flexed knee as well as the extended knee. Further, U.S. Pat. No. 7,468,048 to Meehan 2008, does not describe a pneumatic or non-pneumatic brace that is used on the flexed knee, nor describes a pneumatic or non-pneumatic brace without a window or aperture that can force fluid to areas where it may be accessed without an aperture. This patent also does not describe the role of that device in creating an increased success in obtaining diagnostic fluid (diagnosed as at least 2 ml of fluid) in a non-effusive knee. This patent also does not describe the utility of a compression device in enhancing injecting medication into the joint by dilating the accessible synovial space increasing the target for injecting.

Therefore, there is a need to provide a simple, non-pneumatic, inexpensive device that eases the difficulty in performing a joint needle procedure, thereby increasing the odds of successfully performing the aspiration or needle placement without inadvertently contacting surrounding tissue or bone and accurately placing the needle into the dilated joint space. There is also a need to provide a device that allows general practitioners or other non-specialists to more easily perform the procedure by increasing the size of the membrane area where fluid pools so that the needle may be inserted to penetrate the fluid sac surrounding the joint.

With respect to the prior art discussed above, while a number of non-inflatable and inflatable knee brace configurations are known, none of the prior art devices provide adequate functionality for facilitating a joint aspiration and needle placement procedures for injection using a non-pneumatic, non-inflatable knee brace device that provides focused constant pressure and predetermined areas of low pressure where fluid will pool and expand the synovial membrane. As will become evident, the invention disclosed herein provides advantages for joint procedures similar to those of a tourniquet for facilitating phlebotomy and blood sampling, that is this invention permits synovial fluid and the synovial space to be more accessible to a needle, as a tourniquet similarly makes blood and the intravascular space more accessible to a needle by increasing the pressure and causing the target dilate, making it more accessible to the needle, and similar can be released, so that medication can be injected at lower pressure.

A compressive brace for intraarticular needle procedures is described that provides constant compression. An example embodiment comprises a non-pneumatic releasable compressive planar device or tubular sleeve, a method to apply tension to the device or sleeve comprising a non-stretchable material or stretchable elastomeric material with fasteners and/or external compression straps resulting in displacement and increased intraarticular fluid pressure, a design that displaces fluid within the joint to low pressure areas to be readily accessible by needle or catheter to specific anatomic portals so that the joint can be completely aspirated, tension and pressure can be released so that fluid can be easily injected into the joint, and integration of the device into arthrocentesis, surgical, diagnostic, and joint therapy kits. Some example embodiments contain an integrated window or aperture to allow pooling of fluid and to introduce needles and catheters; other embodiments do not have an intrinsic window and may have pneumatic bladders but still shift fluid to more accessible sites. Other embodiments dilate the patellofemoral joint and may displace the patella-making introduction of the needle more successful. The device is economical and can be disposable, and improves diagnostic arthrocentesis success, arthrocentesis volume, lavage, and improved outcomes for intraarticular joint therapies. The device combined with an injected intraarticular therapy is particularly useful. The main embodiments are designed specifically for the knee, but corresponding device embodiments can be used on many non-knee joints.

In order to overcome the various disadvantages inherent in conducting joint aspiration and intraarticular injection procedures, and to overcome the failure of the prior art to address such needs in the medical field, a joint procedure-facilitating device is provided to enhance the capability of a medical practitioner to successfully perform a joint aspiration procedure (diagnostic arthrocentesis), complete decompression of the joint (therapeutic arthrocentesis), intraarticular injection of the joint with medication or therapy, needle placement for orthopedic surgery and diagnostic imaging. The device is made of an open or closed sleeve of flexible, cloth-like or elastomeric material with or without fasteners of a cinch structure, bands with hook and pile (Velcro), or other fasteners to provide closure, tension, and pressure. The device is designed and positioned to place pressure on specified regions of the joint to thereby create local high pressure and displace joint fluid into a targeted lower pressure location in some embodiments defined by the aperture, window, or access portal or in alternative areas outside of the device, preferably the lateral or medial aspect of the suprapatellar bursa and recesses, the lateral or medial aspect of the patellofemoral joint, the cruciate ligaments, or the lateral or medial femoral condyle. In a preferred embodiment the device displaces fluid from the general joint space to the natural pooling area of the lateral recess of the suprapatellar bursa. In another preferred embodiment, the device displaces fluid from the general joint space and suprapatellar bursa to the synovial reflections over the femoral condyles and cruciate ligaments typically used with a bent (flexed) knee.

In an example embodiment, the device resembles in some respects a traditional elastomeric knee brace, but includes unique structural features to facilitate a successful aspiration procedure. In one example embodiment, an access opening is formed by the device at a targeted location, for example overlying the lateral superior knee, where most commonly orthopedic surgeons access the patellofemoral joint, the lateral recess of the suprapatellar bursa, and the main suprapatellar bursa. The lateral approach has been demonstrated to be the most successful in standard arthrocentesis and joint injections, and this device can be used in improving the success and approaches that the proceduralist already uses. The device can also be placed so that the midpatellar approach can be used as described by Jackson et a (Jackson D W 2002). Similarly, if the practitioner is used to medial approaches, the device can be rotated to facilitate these approaches also. The aspirating needle penetrates the joint at the access opening at the distended lateral recess of the suprapatellar bursa, the main supra patellar bursa or the lateral approach to the patellofemoral joint, usually directed inferiority or medially midpatellar, and the distended bursa and patellofemoral joint with increased dimensions and pressure of the fluid located at the opening greatly enhances the ability of the practitioner to successfully penetrate the fluid compartment to aspirate. The access opening is preferably located either on the lateral side of the knee joint, and is oriented to allow the medical practitioner to specifically access the suprapatellar bursa, patellofemoral joint, cruciate synovial reflections, or femoral condyle from the lateral side or alternatively, rotated 180 degrees to accommodate the corresponding medial approaches. Alternatively, the device can have no aperture, but can displace fluid to other accessible joint portals as known to those expert in the art.

One example embodiment of the present invention incorporates a lightweight, washable fabric composed of cloth or elastomeric material where tension and pressure are provided by closing the sleeve-device with straps or closures, that can be reversibly attached and released during the procedure. A closure element, such as hook and pie material, Velcro, snaps, fasteners, and cincture can be used to secure the device around the joint and apply force to displace fluid within the joint.

An example embodiment of the present invention can incorporate a disposable sterile field dressing to reduce risk of inadvertent inoculation of bacteria into the sterile cavity of the joint. In another example embodiment of the invention, the access opening formed by the device can be used in such as an ultrasound procedure to visualize the tissue located at the access opening or in additional access openings.

In one example embodiment, a single access opening can be provided at lateral location for aspirating a knee joint and simply rotated to accommodate the other knee. In another preferred embodiment, the device and access opening are largely symmetrical so that the same device can be used on both the left and right knee. The portion of the access opening that is not be used can be covered with the binding straps that provide pressure and force to the device and prevent pooling at the unused portion of the aperture.

In another example embodiment, there is no access opening, and the device is placed on the superior knee and compresses the suprapatellar bursa, forcing fluid to the femoral condyles and synovial reflections of the cruciate ligaments, permitting the knee to be aspirated in the flexed knee position (the patient sitting), using the inferior lateral or medial portals, or other classic portals. An example embodiment includes a pneumatic version without a window or access portal.

These and other embodiments share a general characteristic: a releasable pressure device, causing localized high pressure in the synovial fluid with selective locational anatomic displacement of distended joint fluid from distended soft tissues to a targeted low pressure location defined by an access opening or an area outside of the device, most preferably overlying the suprapatellar bursa, the lateral recess of the suprapatellar bursa, the patellofemoral joint, the synovial reflections of the cruciate ligaments, or the synovial reflections of the femoral condyles, depending on the approach, and providing an access opening in the device at the targeted location. This device, appropriately sized, can be used on other joints as well with appropriate size and structure modifications and examples of such embodiments are described.

With a joint aspiration device according to the present invention, the pressure moves fluid from high pressure areas and surrounding soft tissues to low pressure areas, increasing the volume and pressure of the fluid to be aspirated and the effective size of the targeted low pressure area, thereby reducing the chance that the aspirating needle will strike bone or will strike unintended tissue such as nerves or tendons and a greater chance that the needle will enter the intraarticular space. The device can also facilitate complete decompression of the joint, which has been demonstrated to make subsequently intraarticularly injected medication more effective, including corticosteroid and hyaluronate. The device can be made in various sizes to accommodate joint sizes encountered with adults, children, or obese patients, and can be especially functional in a one-size-fits all design for the knee.

The intraarticular pressure and synovial fluid movement are dynamic throughout the phases of the arthrocentesis procedure when an external compressive brace as in the present invention is applied to a knee. There are 3 dynamic phases: (1) the brace is first applied (creating an intraarticular pressure differential), (2) equilibrium (intraarticular pressure homogeneity) is established as fluid moves from the compressed tissues into the synovial fluid space, and (3) arthrocentesis phase (creating an intraarticular pressure differential again between the synovial fluid compartment and the needle opening). When the brace is first applied the low-pressure area is at the portal or access point so fluid flows from high-pressure areas compressed by the brace to low-pressure areas not compressed by the brace. However, after the intraarticular fluid has moved within the joint from high-pressure areas to low-pressure areas, the intraarticular pressure reaches a new equilibrium so that the fluid pressure is the same at the portal or access point as it is in compressed areas covered by the brace—this is the new steady state. However, once the fluid is accessed with a needle at the portal it creates a low-pressure outlet again at the access point, and the system become unstable again so more intraarticular fluid flows from the high-pressure areas compressed by the brace to the low-pressure access point until flow ceases because all of the effusion has been removed.

Ike et al. have demonstrated that voluntary quadriceps contraction can move otherwise occult fluid to the lateral recess of the suprapatellar bursa where the fluid can be accessed in a manner very like the compressive brace [Ike et al 2010]. The reasons for this fluid movement are very similar to those for fluid movement when using a constant compression brace as described herein, that is contraction of the quadriceps muscle creates increased pressure in the inferior knee, the patellofemoral joint, and medial bursa, thereby forcing fluid to the lateral recess of the suprapatellar bursa. A disadvantage to quadriceps contraction technique is that contraction of the quadriceps muscle forces the patella and quadriceps tendon firmly against the femur making the patellofemoral joint completely inaccessible if that were an intended target. Also, it is impossible for the patient to maintain constant contraction (due to the muscle fibers contracting irregularly and intermittently and eventually tiring during contraction), so that the tissues tremble creating an unstable procedure environment with tissue movement against the procedure needle tip causing accentuated pain. The constant compression brace can provide the same benefits as quadriceps contraction but permits the patient to relax the muscles, creates a stable procedure environment, and permits ready access to the patellofemoral joint if that were the desired target.

An unanticipated aspect of these compressive devices in joint injection procedures (intraarticular injection of medication or therapy) is that the response to injected medication is substantially better in both the effusive and non-effusive joints treated with the compressive brace relative to conventional treatment. This fundamental improvement in the outcome of intraarticular injection procedures provides a potential for improvement in the cost-effectiveness of existing intraarticular therapies as well as new integrated systems consisting of a compressive brace and injected intraarticular therapy that have not to date been described.

These and other features and advantages of the invention will become apparent from review of the following drawings, taken in conjunction with the detailed description.

### BRIEF DESCRIPTION OF DRAWINGS

FIG. 1 is a front (anterior) and back (posterior) view of the of the present invention in a first example embodiment.

FIG. 2 is a front (anterior) and back (posterior) view of an alternative embodiment of the present invention in a second example embodiment with one of straps facing in an alternative direction.

FIG. 3 is an example embodiment with a different fastener system, in the case a cinch system.

FIG. 4 is an example embodiment with a tubular structure.

FIG. 5 is an example embodiment with a different shapes and symmetry of access openings, windows, portals, or apertures.

FIG. 6 is an illustration of the non-effusive knee and the synovial surfaces that are targets of arthrocentesis and intraarticular injection procedures.

FIG. 7 is an illustration of the needle approaches that are typically used for arthrocentesis and intraarticular injection procedures

FIG. 8 is an illustration of how the compressive brace in the non-effusive (dry) knee moves the effective synovial fluid and space towards the portal.

FIG. 9 is an illustration of how the compressive brace increases the target area of the synovial membrane in the non-effusive dry knee for needle procedures.

FIG. 10 is an illustration of an application where the window or aperture is placed over the patella and a compressive brace increases the target area of the synovial membrane for the various subpatellar and patellofemoral joint approaches for needle procedures.

FIG. 11 is an illustration of how a compressive brace increases synovial fluid yield in the non-effusive extended knee.

FIG. 12 is a frontal (anterior) view and demonstrates how a compressive brace in the extended effusive knee shifts the bulk of the fluid toward the portal where the fluid can be better accessed.

FIG. 13 is a side (lateral) view that provides an illustration of how a compressive brace in the extended effusive knee shifts the bulk of the fluid toward the portal where the fluid can be better accessed.

FIG. 14 is a frontal (anterior) view that provides an illustration of how the positioning of the aperture and compressive component of the compressive brace in the extended effusive knee shifts the bulk of the fluid toward where ever the portal is positioned.

FIG. 15 is a frontal (anterior) view that provides an illustration of how the diameter of the aperture is critical to permit pooling of synovial fluid.

FIG. 16 is a frontal (anterior) view that provides an illustration of how the fluid can be more easily accessed with a compressive brace.

FIG. 17 is a side (lateral) view that provides an illustration of how a compressive brace in the extended effusive knee shifts the bulk of the fluid toward the portal where the fluid can be better accessed and drained by the needle.

FIG. 18 is an illustration of how the compressive brace increases synovial fluid yield in the effusive extended knee.

FIG. 19 is a frontal (anterior) view that provides an illustration of how a compressive brace can also be used to move fluid in the flexed (bent) knee using the same design also using the lateral suprapatellar approach.

FIGS. 20 and 21 are side (lateral) views that provide illustrations of how a compressive brace can also be used to move fluid in the flexed (bent) knee using the same design also using the lateral suprapatellar approach.

FIG. 22 is a front and back view of a strap-like example embodiment useful specifically for the flexed (bent) knee positioning similar to FIGS. 20 and 21 that provides focused pressure over the suprapatellar bursa permitting non-suprapatellar bursa approaches (medial or lateral patellofemoral or medial inferior approaches) for arthrocentesis.

FIG. 23 is a front and back view of a double parallel strap example embodiment useful specifically for the flexed (bent) knee positioning similar to a conventional knee brace with the inferior portion deleted, which provides focused pressure over the suprapatellar bursa permitting non-suprapatellar bursa approaches (medial or lateral patellofemoral or medial inferior approaches) for arthrocentesis. This example embodiment provides a patella (knee cap) localizer.

FIG. 24 is a front and back view of a double parallel strap example embodiment used specifically for the flexed (bent) knee positioning similar to a conventional knee brace with the inferior portion have deleted which provides focused pressure over the suprapatellar bursa permitting non-suprapatellar bursa approaches (media or lateral patellofemoral or medial inferior approaches) for arthrocentesis. This example embodiment does not provide a patella (knee cap) localizer.

FIG. 25 is a front and back view of a double anti-parallel strap example embodiment useful specifically for the flexed (bent) knee positioning similar to a conventional knee brace with the inferior portion deleted, which provides focused pressure over the suprapatellar bursa permitting non-suprapatellar bursa approaches (media or lateral patellofemoral or medial inferior approaches) for arthrocentesis. This example embodiment provides a patella (knee cap) localizer.

FIG. 26 is a front and back view of a double anti-parallel strap example embodiment useful specifically for the flexed (bent) knee positioning similar to a conventional knee brace with the inferior portion deleted, which provides focused pressure over the suprapatellar bursa permitting non-suprapatellar bursa approaches (media or lateral patellofemoral or medial inferior approaches) for arthrocentesis. This example embodiment does not provide a patella (knee cap) localizer.

FIG. 27 is an opened and folded side view of an continuous ring-like example embodiment useful specifically for the flexed (bent) knee positioning similar to a conventional knee brace with the inferior portion deleted, which provides focused pressure over the suprapatellar bursa permitting non-suprapatellar bursa approaches (medial or lateral patellofemoral or medial inferior approaches) for arthrocentesis.

FIG. 28 is an illustration of how the suprapatellar compressive devices as in FIGS. 22-27 are placed directly above the knee (directly proximal to the knee) and then compress the suprapatellar bursa, forcing fluid to the inferior knee.

FIG. 29 is an illustration of how the suprapatellar compressive devices as in FIGS. 23-26 are placed directly above the knee (directly proximal to the knee) and then compress the suprapatellar bursa, forcing fluid to the inferior knee, showing the strap positioning.

FIG. 30 is an illustration of the use of suprapatellar compressive devices as in FIGS. 23-26 with an alternative fastening mechanism, in this case a cinch with hook and pie.

FIG. 31 is an illustration of example embodiments with air bladders that can be reversibly filed.

FIG. 32 is an illustration of how a suprapatellar brace forces the fluid from the suprapatellar bursa and other knee spaces to the inferior knee, in particular the synovial reflections surrounding the medial and lateral femoral condyles and the synovial reflection of the cruciate ligaments.

FIG. 33 is an illustration of how a suprapatellar brace forces the fluid from the suprapatellar bursa and other knee spaces to the inferior knee, and how the knee can be accessed by the inferior portals including the lateral and medical portals defined by the inferior portion of the patella, the proximal tibia, and medial or lateral patellar tendon respectively.

FIG. 34 is an illustration of how in the non-effusive (dry) knee, a suprapatellar brace forces resident fluid from the suprapatellar bursa and other knee spaces to the inferior knee, and how the dilated synovial spaces in the inferior knee can be accessed.

FIG. 35 is an illustration of how in 140 non-effusive (dry) knees, a suprapatellar brace significantly increased both successful arthrocentesis (at least 2 ml of fluid) and absolute fluid yield in milliliters.

FIG. 36 is an illustration of how in the effusive (swollen) knee, a suprapatellar brace also forces resident fluid from the suprapatellar bursa and other knee spaces to the inferior knee, and how the dilated synovial spaces in the inferior knee can be accessed.

FIG. 37 is an illustration of how in 34 effusive (swollen) knees, a suprapatellar brace significantly increased both successful arthrocentesis (at least 2 ml of fluid) and absolute fluid yield in milliliters.

FIG. 38 is an illustration of variations of the configuration and use of the embodiments of the compressive brace embodiments as shown in FIGS. 22 through 34.

FIG. 39 is an illustration of variations of the configuration and use of the embodiments of the compressive brace embodiments as shown in FIGS. 22 through 34 and in FIG. 38, but in an anterior extended knee view.

FIG. 40 is an illustration of an example embodiment with a window that is suitable for the ankle, which forces fluid to the superficial ankle where it can be aspirated.

FIG. 41 is an illustration of an example embodiment with a window that is suitable for the ankle, demonstrating the movement of fluid from the posterior ankle to the anterior ankle where it can be accessed.

FIG. 42 is an illustration of an example embodiment with a window that is suitable for the wrist.

FIG. 43 is an illustration of an embodiment with a window that is suitable for the wrist fastened into the functional position.

FIG. 44 is an illustration of an embodiment of a compressive brace with a window that is suitable for the wrist demonstrating the movement of fluid from the joint as a whole to the dorsum of the wrist where it can be accessed.

FIG. 45 is a front (anterior) and back (posterior) view of an example embodiment where there is no window or aperture in the brace per se, rather when the brace is wrapped around the knee the low pressure window or aperture is formed by the edges of the internal straps and body.

FIG. 46 is a front (anterior) and back (posterior) view of an example embodiment where there is no window or aperture in the brace per se, rather when the brace is wrapped around the knee the low pressure window or aperture is formed by the internal edges of the straps and body.

FIG. 47 is a front (anterior) view of the knee with a compressive brace of the embodiments of FIG. 45 and FIG. 46. in place showing the concentration of fluid in the patellofemoral joint so that traditional approaches to arthrocentesis and injection can be utilized.

FIG. 48 is a front (anterior) view of the knee with a compressive brace of the embodiments of FIG. 45 and FIG. 46 in place showing that the compressive brace displaces and lifts the patella off of the femur permitting an effective increase in joint space permitting more facile passage of the procedure needle.

FIG. 49 is an illustration of results showing that, when used in 430 subjects, 215 with a constant compression brace and 215 without a constant compression brace, the constant compression group has a longer time to next injection (time until next symptomatic joint flare) thus increasing the effectiveness and duration of the injected medication and increasing the cost-effectiveness of injection procedure.

FIG. 50 is an illustration of results showing that, when used in 76 subjects with clinically effusive (swollen) knee, 38 with a constant compression brace and 38 without a constant compression brace, the constant compression group has a longer time to next injection (time until next symptomatic joint flare) thus increasing the effectiveness and duration of the injected medication and increasing the cost-effectiveness of injection procedure.

FIG. 51 is an illustration of results showing that, when used in 354 subjects with clinically non-effusive (dry) knee, 177 with a constant compression brace and 177 without a constant compression brace, the constant compression group has a longer time to next injection (time until next symptomatic joint flare) thus increasing the effectiveness and duration of the injected medication and increasing the cost-effectiveness of injection procedure.

FIG. 52 is an illustration of results showing that, when used in 61 subjects with osteoarthritis of the knee, 19 with a constant compression brace and 42 without a constant compression brace, the constant compression group has a longer time to next injection (time until next symptomatic joint flare) thus increasing the effectiveness and duration of the injected medication and increasing the cost-effectiveness of injection procedure.

### DESCRIPTION OF EMBODIMENTS AND INDUSTRIAL APPLICABILITY

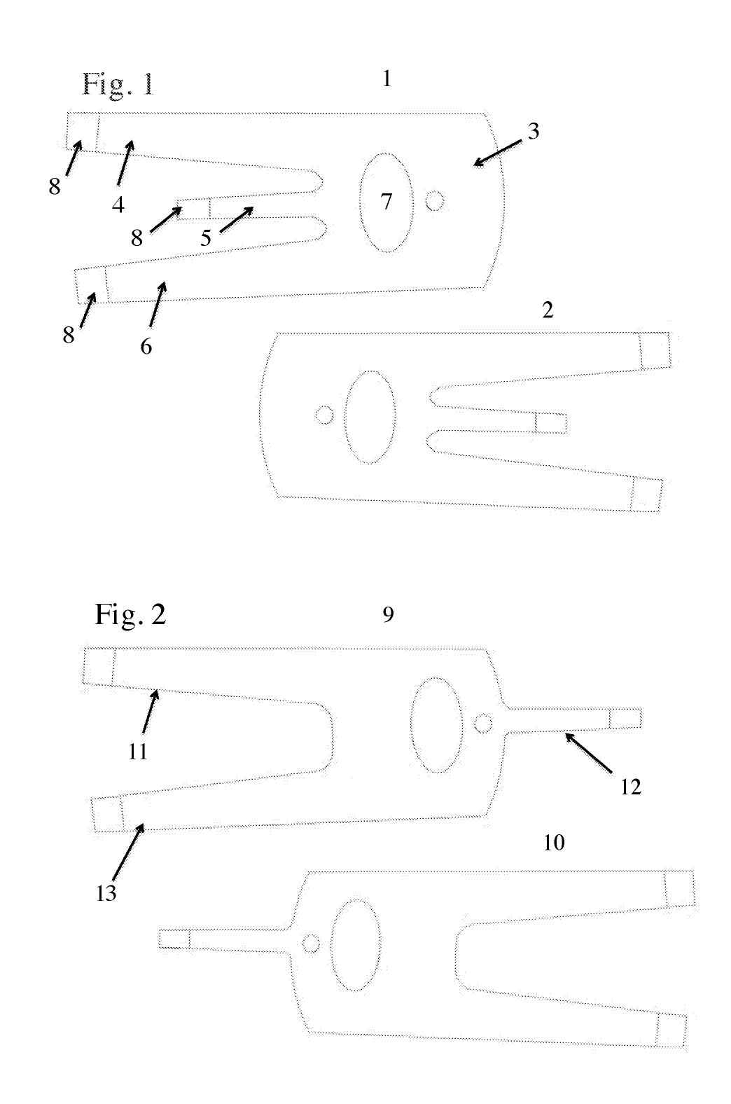

FIG. 1 illustrates a first example embodiment of a joint aspirating device of the present invention, showing a front 1 and back 2 view. In this embodiment, the device is used for facilitating aspiration and injection of the knee joint. The device includes a main panel or body 3 made of a flexible material that is suitable to be wrapped around the joint when in use. Straps 4, 5, and 6 are used to close, tighten, and secure the device to the opposite side to provide tension to selectively apply pressure to the joint in order to cause high pressure joint fluid displacement to a low pressure targeted location at a window or aperture 7, in this embodiment examples including the suprapatellar bursa, lateral recess, and patellofemoral joint, by fastening to other parts of the device usually the forward portion or anywhere on 3 where there is receptive material for fastening. The straps can be from one to three in number (4-6 as shown), and can be any number. The material is flexible and can be elastomeric/elastic, since it is desirable to compress the joint so that the device displaces the joint fluid with or without deformation or stretching of the main panel. The synovial fluid moves from areas of high pressure (areas compressed by the compression device) to areas of low pressure (areas not contacted by the compression device) in this example, the access opening or aperture 7. The opposite longitudinal side is defined by straps (in this case, 4, 5, and 6) that are used to secure the device to its opposing side 3 and to the patient and selected joint. The straps 4, 5, and 6 include securing elements, such as a male/female interdigitating structures, for example hooks or other fasteners 8, that attach to corresponding pie material (such as Velcro fasteners) or other fasteners located on the backside of the main panel or anywhere on 3. The straps 4, 5, and 6 can be parallel as shown in FIG. 1. The window 7 can be elliptical or other shape but can expose most favorably the lateral suprapatellar bursa and/or midpatellar and can also fully expose the patella so the patellofemoral joint, the target of most intraarticular injections, would be dilated with synovial fluid.

Reverse side 2 in FIG. 1 shows the reverse side of the same embodiment as 1, and can comprise hook/pile material (Velcro) or other forms of fasteners that mate with appropriate surfaces and structures on the body or any surface that is appropriate.

FIG. 2 shows an example embodiment in the front and back views that is similar to the embodiment as in FIG. 1, but one or more of the straps 11, 12, and 13 (in this case strap 12) face the opposite direction; the straps can be fewer or greater in number, and there can be no individual straps but solid material with fasteners instead of straps, and will fulfill the same purpose to wrap around the joint and bind the opposing sides the device to create a substantially tubular structure around the joint. These embodiments can be fastened for pressure during aspiration procedures, and can be released to reduce pressure for injection procedures, or alternatively fastened and released.

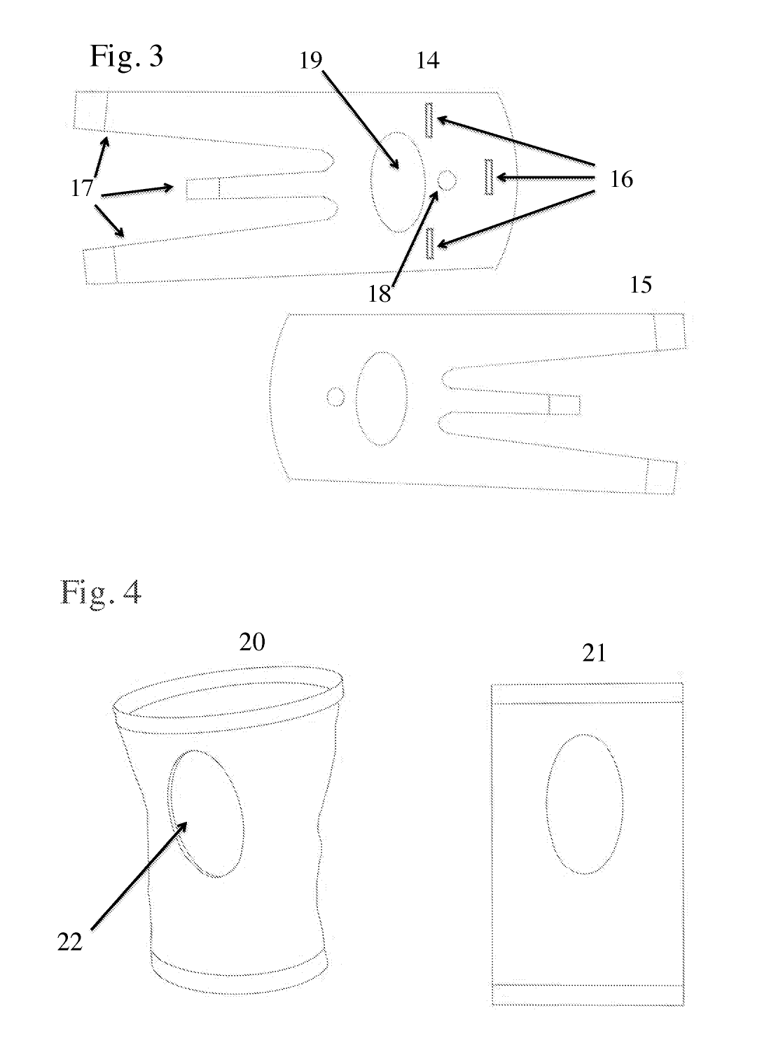

FIG. 3 shows an example embodiment similar to that shown in FIG. 1 with a front view 14 and a back view 15, but instead of the fastener being simple hook and pie, the example embodiment includes cinch devices (16) so that the straps (17) loop through the cinches and then hook to the pie of the same strap or the body of the device or both.

The embodiments in FIGS. 1-3 can have a placement or locator marker 18 (FIG. 3) or surface marker on the device, a separate cutout on the device, or extension, cutout or structure of the access portal that can be used to place the device and access portal in a reproducible anatomic position in relation to underlying palpable or visible anatomy. A particularly useful embodiment of the placement or locator marker consisting of a surface marker, cutout, or extension of the access portal is used to place the device and access portal reproducibly in relation to the patella (knee cap), where the marker to be centered over the center of the midpatellar or the proximal surface of the patella in order to place the portal or access opening 19 at the patellofemoral joint, suprapatellar bursa, and/or lateral recess of the suprapatellar bursa. Another particularly useful embodiment provides for a marker to be centered over the center of the midpatellar or the distal surface of the patella in order to place the portal or access opening at the inferior patellofemoral joint, or the lateral and medial inferior portals of the knee.

The device can also be formed as a unitary tubular sleeve with an access portal with or without tightening straps as described above. FIG. 4 is an example embodiment of the device comprising a unitary tubular sleeve in the expanded view 20 and a collapsed view 21 with an access portal 22, and can be with or without tightening straps as described above. The embodiment as shows is made of elastic material without tightening straps, but these straps can be included and are anticipated by the present invention.

Particularly useful embodiments of all the above example compressive brace devices for the knee can be 5 cm to 45 cm in width to accommodate the different knee areas to be compressed and 10 cm to 100 cm in length or circumference to accommodate different sizes of knees depending on the elastomeric nature and stretchability/contractile aspect or lack thereof of the device material.

FIGS. 1-4 show embodiments that are largely symmetrical and thus may be used on either the left or right leg, therefore, the access opening as shown is symmetrical and can be used on either leg. On the same leg, the device can be rotated medially prior to fastening to instead access the medial side, but as noted the lateral approach is more successful in usual practice. Thus, both medial and lateral sides of the knee joint and both the left and right legs can be exposed for aspiration through the selected position of the access opening as shown. The unused portion of the aperture or access opening can be covered with one of the binding straps if desired.

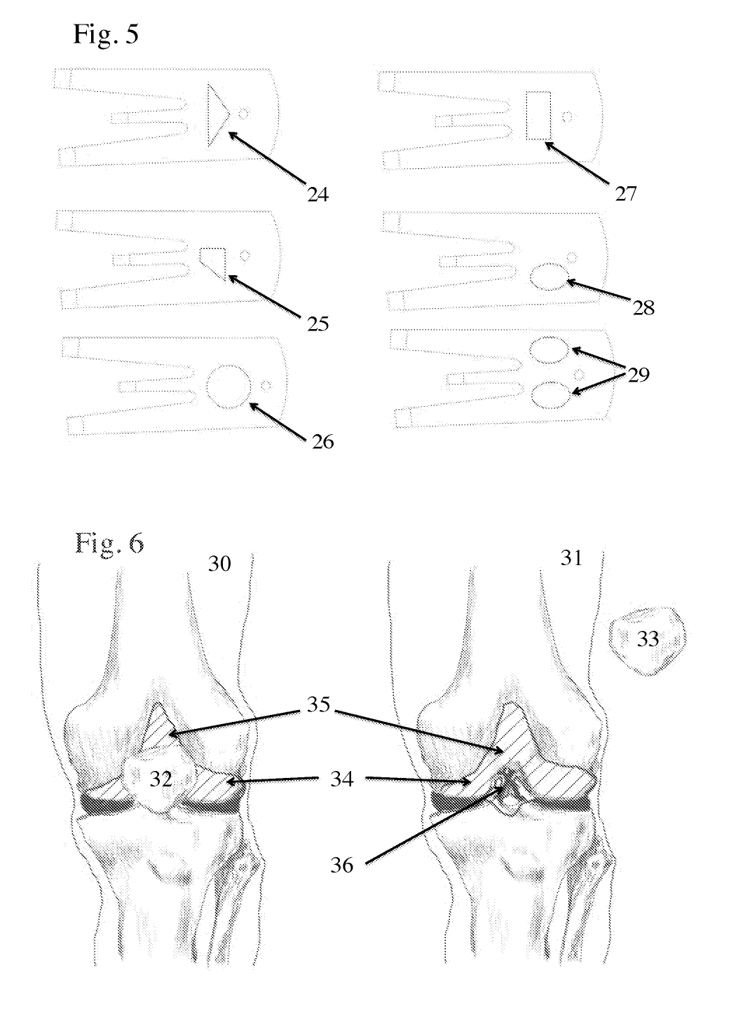

FIG. 5 is a front view of example embodiments where the aperture takes different positions and shapes. The aperture can be any shape including as shown, triangular 24, asymmetrical 25, circular 26, rectangular 27, oval 28, duplicate and symmetrical 29, or other shapes. The aperture, access portal, or window is at least in one dimension from 0.5 cm to 20 cm. The aperture(s) can also be closed or covered with a device that fits over the aperture and binds to the rest of the device with hook and pile fasteners.

FIG. 6 demonstrates the non-effusive (dry) extended knee and the synovial surfaces that are targets of arthrocentesis and intraarticular injection procedures. 30 and 31 are anterior views of the knee, 32 is the patella (knee cap) in anatomic position and 33 the patella has been removed to show the target surfaces 34 of the synovial membrane and joint surfaces of the anterior knee marked as a diagonal hatch 34. As can be seen, although the knee is a large structure, the target structures 34 are extremely anatomically limited for successful needle placement in the intraarticular space as defined by the internal limits of the synovial membrane and the joint surfaces 34 including but not limited to the patellofemoral joint 35 and the synovial reflections of the cruciate ligaments 36.

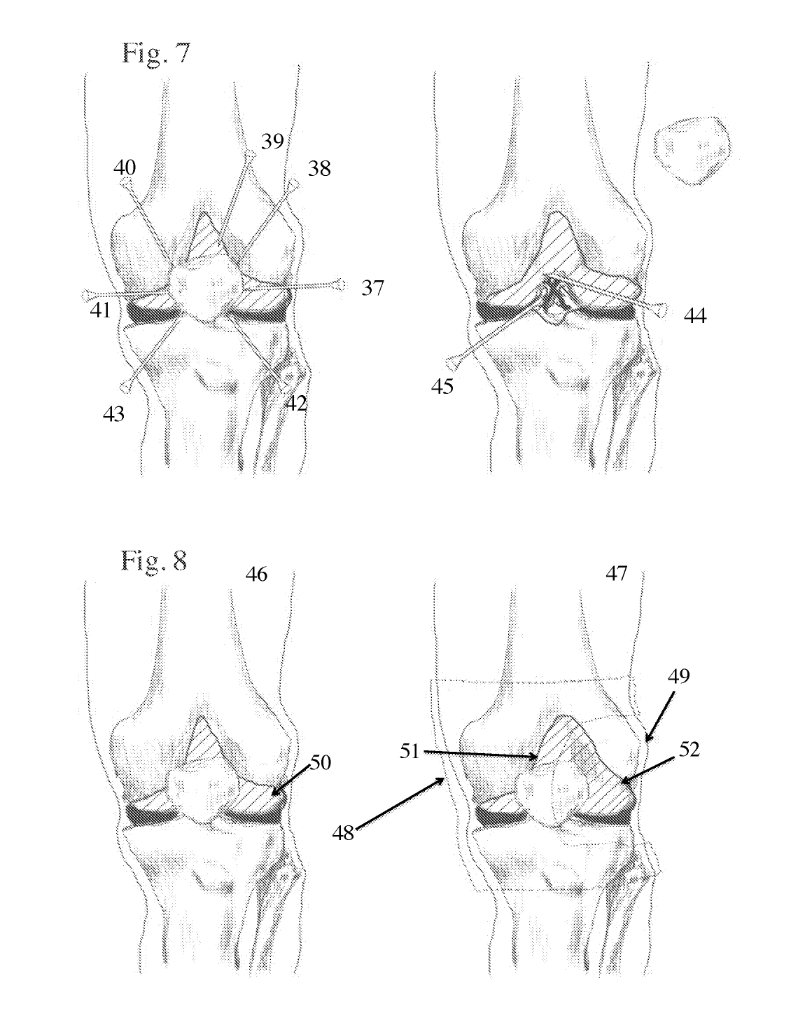

FIG. 7 demonstrates the needle approaches that are typically used for arthrocentesis and intraarticular injection procedures. 37 is the lateral midpatellar approach that has high success and accuracy as reported by Jackson et al (Jackson D W 2002). 38 is the lateral superior approach where the needle again ends up in the patellofemoral joint, the approach that most clinical orthopedic surgeons use, the results of Jackson et al withstanding (Jackson D W 2002). 39 is a similar superior lateral approach that targets the suprapatellar bursa and superior patellofemoral joint. 40 is similar medial superior approach that often transverse the vastus medialis and are generally less successful than 37 and 38. 41 is the medial midpatellar approach that is less successful than the lateral midpatellar approach 37 (Jackson D W 2002). 42 and 43 are the lateral and medial respectively inferior approaches that again target the patellofemoral joint, but often encounter the subpatellar fat pad and are then less successful. 45 utilizes the inferior medial portal and targets the synovial reflections of the cruciate ligaments, and is generally inferior to the lateral midpatellar approach 37 (Jackson 2002). 44 utilizes the lateral inferior portal and targets the synovial membrane and cartilage surfaces of the medial femoral condyle (modified lateral inferior approach) and has been shown to be clinically equivalent to the lateral midpatellar approach (Chavez-Chiang C E 2011).

FIG. 8 demonstrates how a compressive brace in the non-effusive (dry) knee moves the effective synovial fluid and space towards the desired portal. 46 is an anterior view of the non-effusive (dry) knee without the compressive brace and 47 is an anterior view of the non-effusive (dry) knee with the compressive brace. The compressive brace 48 (as defined by dotted line) is oriented so that the window or aperture 49. In the non-compressed knee 46 the target synovial and cartilage surfaces that are not collapsed are limited, thus the target area (diagonal hatch area) 50 is largely symmetrical in regards to the medial and lateral knee. However, when the compressive brace 48 is applied, the synovial space on the medial side collapses 51 while the synovial space and whatever synovial fluid is present shifts to the lateral side greatly expanding the synovial target (diagonal hatch) for the needle 52 that shifts to the window or aperture 49 since there is not pressure at that spot.

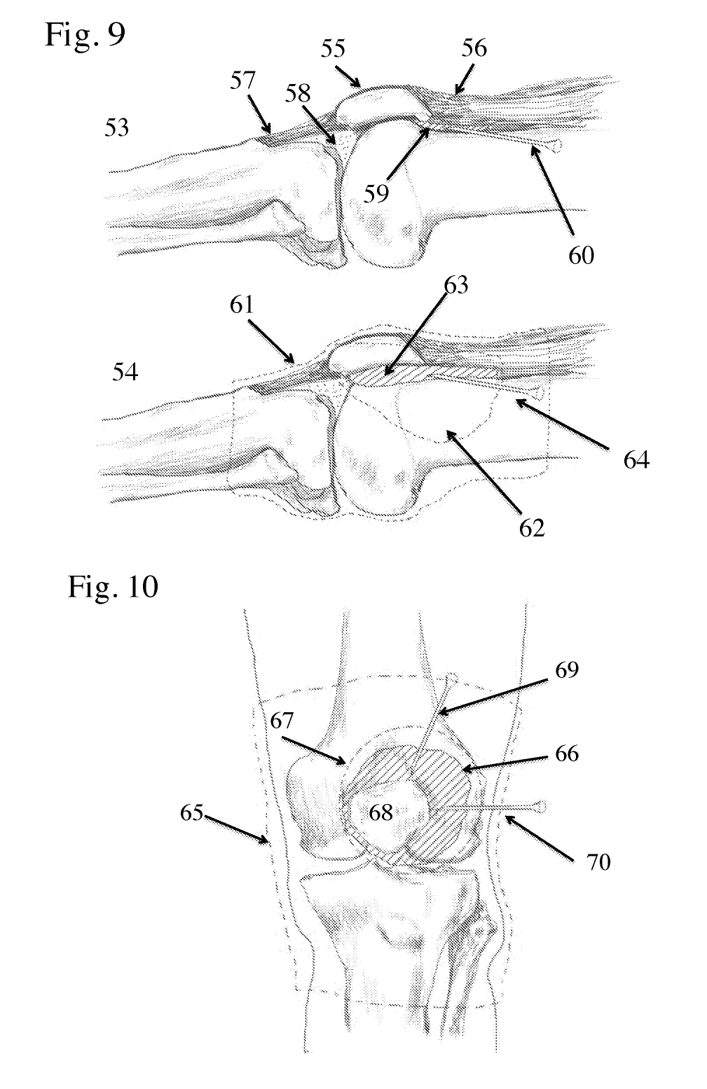

FIG. 9 is a lateral view of the knee and shows how the compressive brace increases the target area of the synovial membrane in the non-effusive dry knee for needle procedures. 53 is the non-effusive (dry) knee in the lateral view without the compressive brace and 54 is the non-effusive (dry) knee in the lateral view with the compressive brace. 55 is the patella (knee cap), 56 is the quadriceps tendon and muscle, 57 is the patellar tendon, 58 is the subpatellar fat pad, 59 is the synovial space target beneath the patella 55 and patellar tendon 56 consisting of the synovial space and synovial fluid (diagonal hatch) of the suprapatellar bursa and patellofemoral joint that is targeted by the needle 60. 61 is the brace (defined by dotted line) with the window or aperture 62 oriented to the lateral superior portal so that the suprapatellar bursa was exposed. It is anticipate the window or portal could fully accommodate the patella so that the suprapatellar joint would dilate with fluid creating a perfect target 63 for both aspiration and injection. With pressure on the medial knee, the medial synovial structure is collapsed and extant synovial fluid is shifted towards the lateral synovial structures and suprapatellar joint under the patella, quadriceps tendon, and suprapatellar bursa, greatly expanding the dimensions of the synovial target 63 for the procedure needle 64.

FIG. 10 illustrates an example case where the window or aperture is placed over the patella and a compressive brace increases the target area of the synovial membrane for the various subpatellar and patellofemoral joint approaches for needle procedures. The brace 65 (broken line) compresses the joint forcing the fluid 66 (diagonal hatch) towards and within the window 67 that surrounds the patella 68 and distal portion of the quadriceps tendon and proximal patellar tendon, causing the patellofemoral joint to become engorged with extant fluid 66 (diagonal hatch). The two most common needle approaches, the lateral superior patellofemoral joint approach 69 and the lateral medial midpatellar approaches can then be undertaken with greater success. If the window 67 and brace 65 are rotated slightly to the medical side, then the corresponding medial approaches to the patellofemoral joints can be undertaken. This embodiment and use can be particularly useful for joint injections, such as corticosteroids, hyaluronic acid derivatives, and other medications or factors.

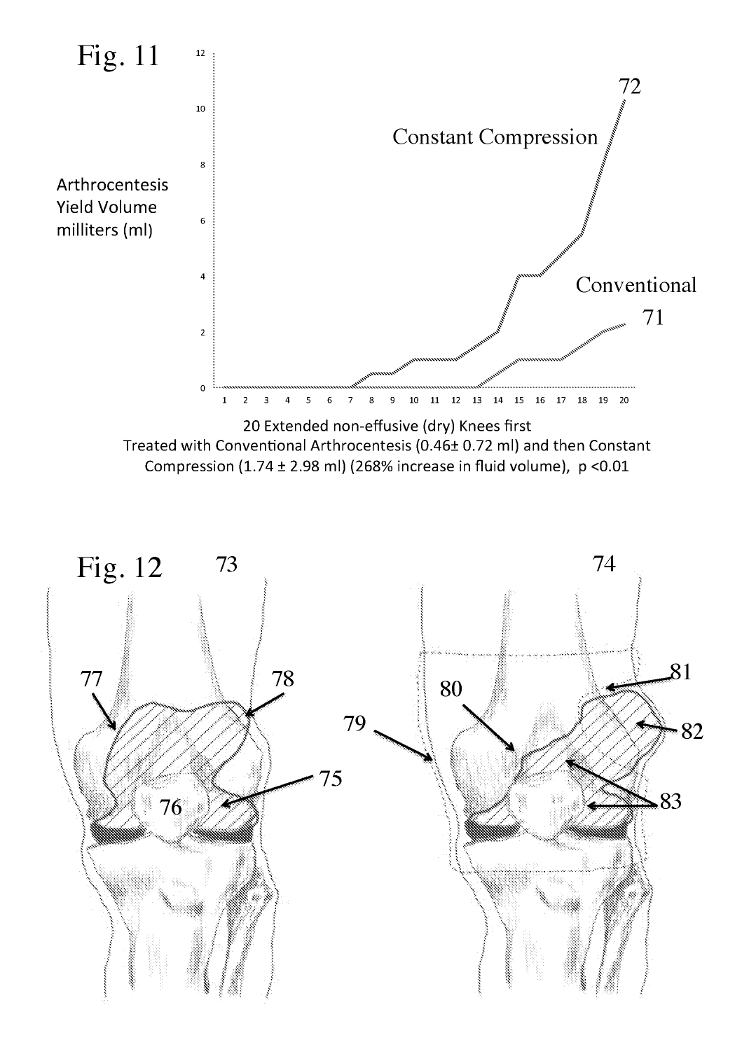

FIG. 11 demonstrates how the compressive brace of the designs in FIGS. 1-10 increases synovial fluid yield in the non-effusive (dry) extended knee using the lateral superior approach in 20 subjects with non-effusive (dry) knees where first conventional arthrocentesis was attempted and fluid volume measured, and then the compressive applied and total fluid volume measured. The lower line 71 shows arthrocentesis yield without the compressive brace and the upper line 72 shows arthrocentesis yield with the compressive brace. As can be seen, the compressive brace markedly increases arthrocentesis success and yield. The compressive device increases successful diagnostic arthrocentesis (at least 0.25 ml of synovial fluid) in the dry knee from 35% to 65% (p 0.06), increases absolute diagnostic arthrocentesis (at least 2.0 ml of synovial fluid) from 5% to 30% (p 0.08), and increases fluid yield by 278% (0.46±0.72 ml without the device to 1.74±2.98 ml with the device (p=0.01). This improvement in needle placement and access to fluid for analysis is important and since the volumes of corticosteroid and hyaluronate injected into the joint (1 to 6 ml) are small, an aspiration of an additional 1 ml in the dry knee may have significant beneficial effect on drug concentration in the dry knee, increasing intraarticular drug concentrations by 15% to 50%. Further, return of synovial fluid proves the intraarticular positioning of the needle, thus, permitting more not only more complete arthrocentesis but also more accurate placement of medication by intraarticular injection.

FIG. 12 is a frontal (anterior) view and demonstrates how a compressive brace according to the present invention in the extended effusive knee shifts the bulk of the fluid toward the portal where the fluid can be better accessed. 73 is an anterior view of the extended knee with effusion and 74 is an anterior view of the extended knee with a brace. In the effusive extended knee without the brace 73 the synovial fluid (diagonal hatch) 75 pools superior to the patella 76 in the suprapatellar bursa both medially 77 and laterally 78, with more fluid moving to the lateral suprapatellar bursa 78. After the compressive brace (broken line) 79 is applied, the medial suprapatellar bursa 80 collapses from the external pressure of the brace 79 and fluid from the non-window side moves over to the window 81 and dilates the lateral suprapatellar bursa 82 and patellofemoral joint 83.

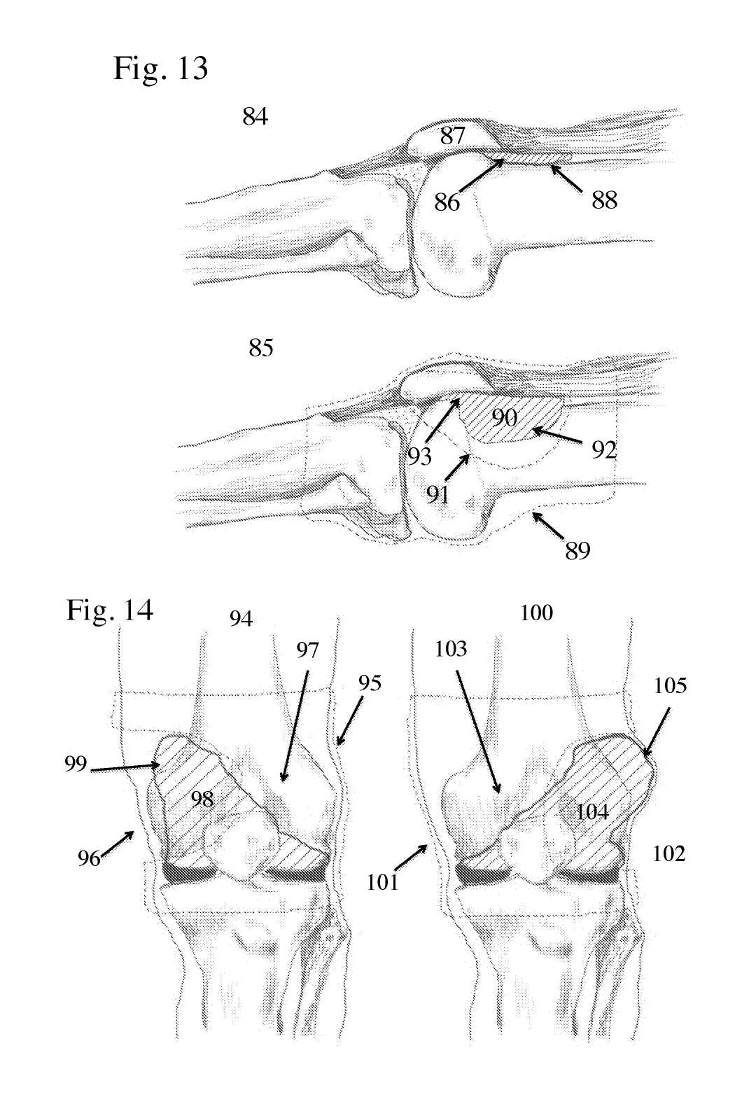

FIG. 13 is a side (lateral) view and demonstrates how a compressive brace according to the present invention in the extended effusive knee shifts the bulk of the fluid toward the window or portal where the fluid can be better accessed. 84 is an anterior view of the extended knee with effusion and 85 is an anterior view of the extended knee with a brace. In the effusive extended knee without the brace 84 the synovial fluid (diagonal hatch) 86 pools superior to the patella 87 in the suprapatellar bursa 88 both medially and laterally. After the compressive brace (broken line) 89 is applied to the extended effusive knee 85, the medial suprapatellar bursa collapses from the external pressure of the brace 89 and fluid (diagonal hatch) 90 from the non-window side moves over to the window 91 and dilates the lateral suprapatellar bursa 92 and patellofemoral joint 93.

FIG. 14 is a frontal (anterior) view and demonstrates how the positioning of the aperture and compressive component of a compressive brace according to the present invention in the extended effusive knee shifts the bulk of the fluid toward where ever the portal is positioned. 94 is anterior view of an effusive (swollen) knee with the compressive brace 95 in place with the window 96 on the medial side of the knee, collapsing the lateral suprapatellar bursa 97, and forcing the synovial fluid (diagonal hatch) 98 to the medial suprapatellar bursa 99. 100 is anterior view of an effusive (swollen) knee with the compressive brace 101 in place with the window 102 on the medial side of the knee, collapsing the medial suprapatellar bursa 103, and forcing the synovial fluid (diagonal hatch) 104 to the lateral suprapatellar bursa 105. Thus, orientation of the brace and/or location of the window and the presence of collapsible or expandable synovial structures under the brace or window determine where synovial fluid will pool and be accessible to the procedure needle.

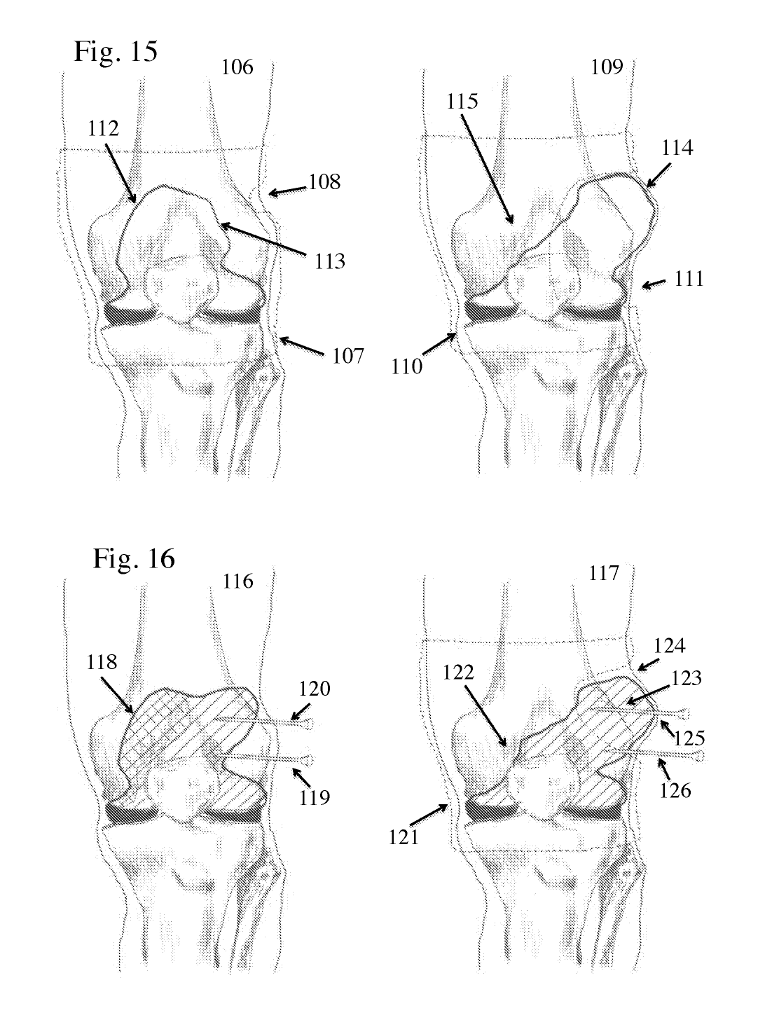

FIG. 15 is a frontal (anterior) view and demonstrates how the diameter of the aperture is critical to permit pooling of synovial fluid. A guiding principle for a portal to permit pooling in a low pressure synovial structure deep to portal or window is that the portal has to be approximately the same magnitude of size or larger than the underlying structure, so that the overlying skin and connective tissue do not have tension and thus pressure and the underlying desired structure can dilate with displaced fluid. Thus, a “window or portal” in a compressive brace according to the present invention does not simply provide access for a needle, but must also be large enough to create a low pressure area comparable in size to the structure intended to be dilated. Thus, the portals must be of adequate dimension or diameter so that underlying structure does not feel pressure transmitted by the brace directly next to the portal and thus can expand and dilate. 106 is a frontal anterior view of an extended leg with a brace 107 with a small window 108, and 109 is a frontal (anterior) view of a compressive brace 110 with a large window 111. The braces 107 and 110 are otherwise identical except for the size of the windows or apertures 108 and 111. The anterior view of the extended knee 106 with the small window 108 of brace 107 has the synovial space of the knee defined by the synovial reflections, cartilage surfaces, and suprapatellar bursa (defined by bold line) 112. As can be seen in 106, although the window 108 is open and provides a defined area of low pressure, the window is too small to permit the overlying skin and connective tissue to stretch and accommodate the much larger structure of the lateral suprapatellar bursa 113, thus, the suprapatellar bursa does not “feel” the small area of transmitted low pressure on the surface and does not dilate, nor does the opposite side 112 collapse. In contrast, as in shown in 109, the large window 111 creates a larger area of low pressure where the overlying skin and connective tissue can stretch, and the large underlying structure, the lateral suprapatellar bursa 114 can dilate and expand into this low pressure area created by the window 111, while the opposite areas under high pressure, including the medial suprapatellar bursa 115, collapse as the low pressure area, the lateral suprapatellar bursa 114, dilates. Thus, this demonstrates the crucial relationship between window and portal size and the size of the intended structure to be dilated and thus accessed. In the superior knee, the window or portal can be at least 2 to 10 cm in diameter, with optimal diameter at 4 to 8 cm. In the inferior knee, because of the constrained anatomy the portals can be smaller, or not exist at all as we describe later in more detail.

FIG. 16 is a frontal (anterior) view of an extended knee without 116 and with a compressive brace 117 and demonstrates how the fluid can be more easily accessed with a compressive brace according to the present invention. In the knee without a brace 116, the fluid in the medial knee and medial suprapatellar bursa (cross hatch area) 118 is relatively inaccessible to the superior 119 and inferior 120 needles. In contrast, in the knee 117 with a compressive brace 121, the medial synovial structures that were previously inaccessible have collapsed 122, and the fluid has shifted to the lateral patellofemoral joint and the lateral suprapatellar bursa (diagonal hatch) 123 permitted by the low pressure area defined by the large window 124, and are thus, much more accessible to the superior 125 and inferior needles 126.

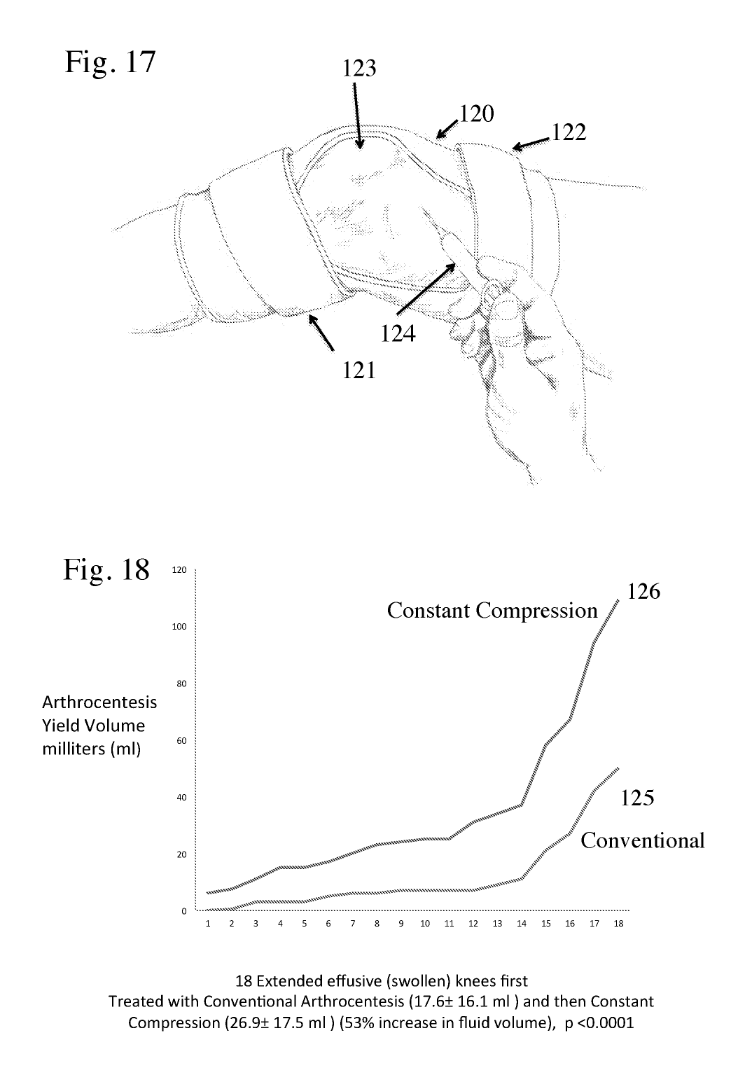

FIG. 17 shows at lateral view with a brace 120 of the design of FIGS. 1-5 affixed to an extended knee with a compressive mechanism, in this case straps 121 and 122, and a window or portal 123 large enough to permit the suprapatellar bursa and/or patellofemoral joint to dilate with synovial fluid, that can be then accessed with a needle and syringe 124.

FIG. 18 is an illustration of results showing how a compressive brace as in the embodiments in FIGS. 1-10 increases synovial fluid yield in the effusive (wet) extended knee using the lateral superior approach in 18 subjects with non-effusive (dry) knees. The lower line 125 shows arthrocentesis yield without a compressive brace and the upper line 126 shows arthrocentesis yield with a compressive brace according to the present invention. As can be seen, the compressive brace markedly increases arthrocentesis success and yield. The compressive device increases absolute diagnostic arthrocentesis (at least 2.0 ml of synovial fluid) from 72% to 100% (p 0.004), and increases fluid yield by 53% (17.6±16.1 ml without the device to 26.9±17.5 ml with the device (p=0.0001). This improvement in needle placement and access to fluid for analysis is important and since the volumes of corticosteroid and hyaluronate injected into the joint (1 to 6 ml) are small, an aspiration of an additional 9.3 ml in the effusive knee may have significant beneficial effect on drug concentration in the effusive knee, increasing intraarticular drug concentrations by 50% to 90%.

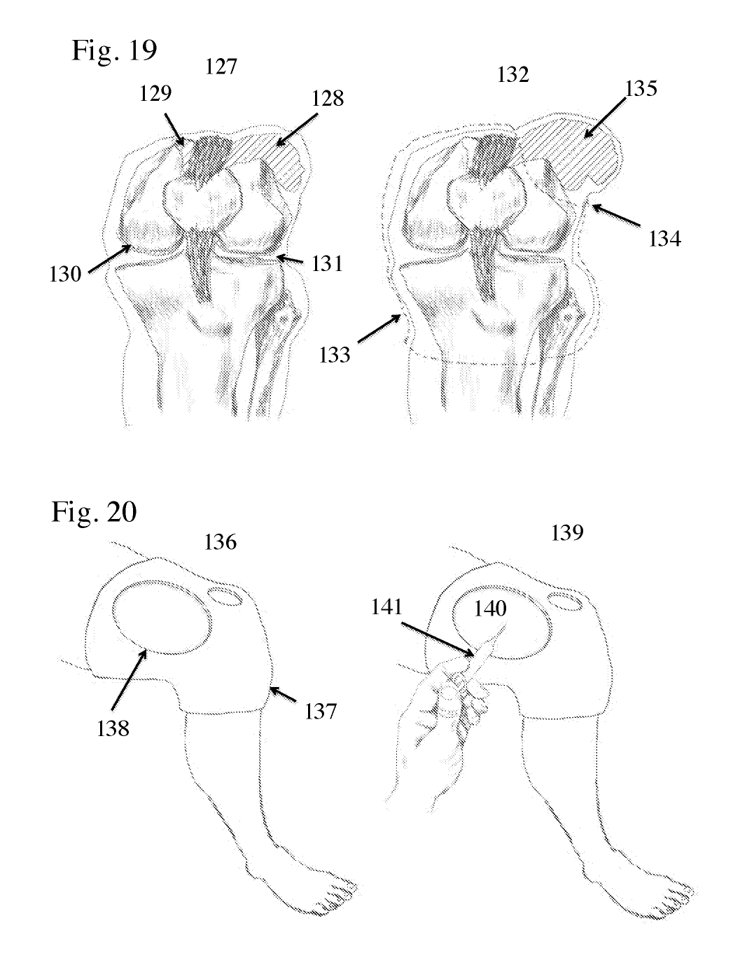

FIG. 19 is a frontal (anterior) view and demonstrates how a compressive brace according to the present invention can also be used to move fluid in the flexed (bent) knee using the same design also using the lateral suprapatellar approach. 127 is a frontal (anterior) view of a flexed knee without a brace. In this position most of the fluid shifts to the lateral suprapatellar bursa (diagonal hatch) 128, but fluid remains in the patellofemoral joint (diagonal hatch) 128, overlying the medial femoral condyle 130 and lateral femoral condyle 131 as well as the other synovial membrane reflections and joint surfaces. 132 is a frontal (anterior) view of a flexed knee with a brace (broken line) 133. As in the extended knee, the compressive brace 133 forces the fluid to the area of the low pressure window 134 and dilates the synovial structure in this window, in this case the lateral superior suprapatellar bursa 135

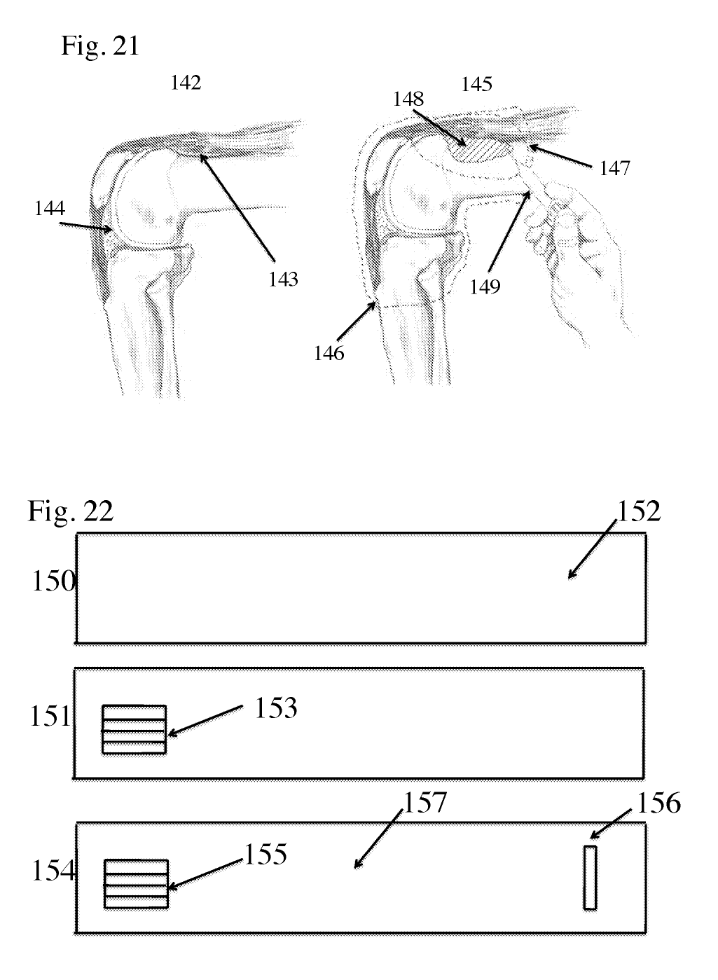

FIGS. 20 and 21 are side (lateral) views and demonstrate how a compressive brace according to the present invention can also be used to move fluid in the flexed (bent) knee using the same design also using the lateral suprapatellar approach. FIG. 20 shows the side (lateral) view 136 of the flexed (bent) knee with the brace 137 in place and the window 138 oriented to the lateral suprapatellar bursa. 139 is a side (lateral) view showing the lateral suprapatellar bursa 140 being accessed with a needle and syringe 141.

FIG. 21 shows the side (lateral) view 142 of the flexed (bent) knee without a brace, showing fluid in the suprapatellar bursa (diagonal hatch) 143, overlying the femoral condyles (diagonal hatch) 144 and other joint surfaces, a brace 137 in place and the window 138 oriented to the lateral suprapatellar bursa. 139 is a side (lateral) view showing the lateral suprapatellar bursa 140 being accessed with a needle and syringe 141. 145 is a lateral view of the flexed knee with the compressive brace (broken line) 146. The window 147 provides a low-pressure area, and the fluid shifts to the lateral suprapatellar bursa 148 where it can be accessed by needle and syringe 149.

FIG. 22 is a front and back view of a strap-like example embodiment suitable specifically for the flexed (bent) knee positioning similar to FIGS. 20 and 21 that provides focused pressure over the suprapatellar bursa permitting non-suprapatellar bursa approaches (medial or lateral patellofemoral or medial inferior approaches) for arthrocentesis.

FIG. 22 is a back (dorsal) view 150 and skin side (front) view 151 of an example strap-like embodiment useful typically for the flexed (bent) knee positioning, that provides focused pressure over the suprapatellar bursa permitting non-suprapatellar bursa and non-patellofemoral approaches for arthrocentesis and joint injection with similar flexed knee positioning as in FIG. 20-21, but there is only one strap, not multiple straps or even a window or aperture, thus, the device can be very simple. The strap 150 can be affixed in a number of ways; one efficient approach uses a pile surface 152 that binds to the undersurface of the strap 151 by means of hooks (e.g., Velcro) 153 that bind to the pile surface 152. Alternatively, a cinch embodiment 154 can be used with a hook surface (Velcro) 155, that is looped through the cinch 156 and bonds with the pile 157 reversibly bind the strap to the superior knee permitting successful arthrocentesis from the inferior portals. This embodiment, as an example, would be useful in dimension of 1 cm to 25 cm in width and 10 cm to 100 cm in length.

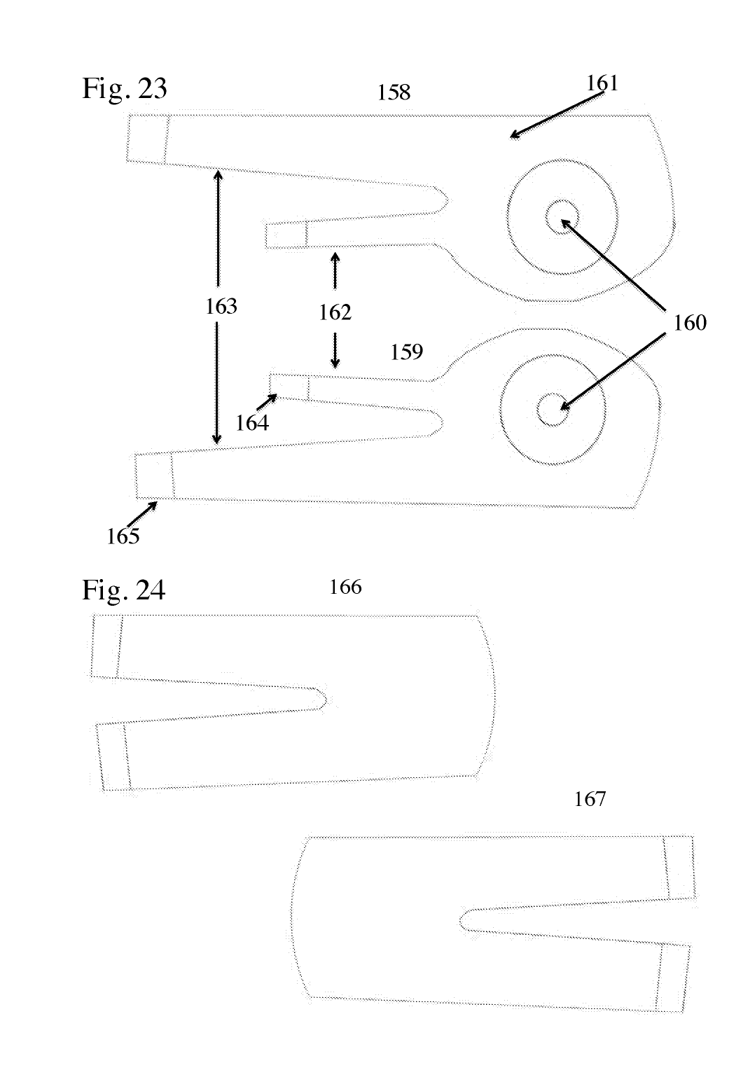

FIG. 23 is a front 158 and back 159 view of a double parallel strap example embodiment useful specifically for the flexed (bent) knee positioning similar the device shown in FIG. 22 and also similar to a conventional knee brace with the inferior portion deleted to make it more narrow. This embodiment provides focused pressure over the suprapatellar bursa permitting non-suprapatellar bursa approaches (medial or lateral patellofemoral or medial or lateral inferior approaches) for arthrocentesis and joint injection This version provides a patella (knee cap) localizer 160.

The example embodiment includes a main panel or body 161 comprising a flexible material that is suitable to be wrapped around the joint Straps 162 and 163 are used to close, tighten, and secure the device to the opposite side to provide tension to selectively apply pressure to the joint in order to cause high pressure joint fluid displacement to a low pressure targeted location, in this embodiment for example the patellofemoral joint, the femoral condyles, or the synovial reflections of the cruciate ligaments. In this example embodiment there is no window or aperture because the low-pressure area is outside of the brace, thus a separately defined window is not necessary but the overall shape provides the same beneficial fluid displacement effect Pressure is provided to the joint by fastening to other parts of the device, usually the forward portion or anywhere on 161 where there is receptive material for fastening. The straps can be from one to three in number (2 as shown), but there can be any number of straps. Preferably, the material is flexible and could elastomeric/elastic, since it is desirable to compress the joint so that the device displaces the joint fluid with or without deformation or stretching of the main panel. The synovial fluid moves from areas of high pressure (areas compressed by the compression device) to areas of low pressure (areas not contacted by the compression device. The opposite longitudinal side is defined by straps (in this case 161 and 162) that are used to secure the device to its apposing side 161 and to the patient and selected joint. The straps 162 and 163 include securing elements, such as a male/female interdigitating structures for example hooks or other fasteners 164 and 165 that attaches to corresponding pile material (such as Velcro fasteners) or other fasteners located on the backside of the main panel or anywhere on 161.

FIG. 24 is a front 166 and back 167 view of a double parallel strap example embodiment useful specifically for the flexed (bent) knee positioning similar to the embodiment in FIG. 23 with all the same structural features and straps and fasteners, but version does not provide a patella (knee cap) localizer. This embodiment would be used similarly as the embodiments of FIGS. 22 and 23.

FIG. 25 is a front 168 and back 169 view of a double anti-parallel strap example embodiment useful specifically for the flexed (bent) knee positioning similar in structure and function to embodiments shown in FIGS. 22-24. This version provides a patella (knee cap) localizer 170, and straps 171 and 172 that are antiparallel. This embodiment includes the fasteners 173 and 174, that can be hook and pie, or other fasteners as described earlier.

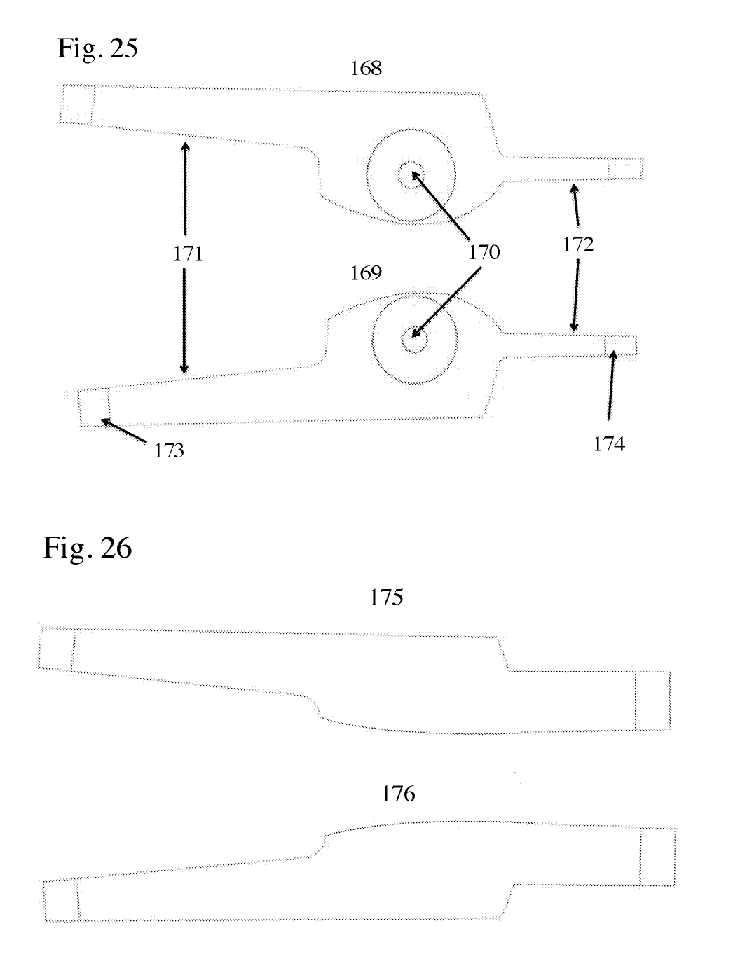

FIG. 26 is a front 175 and back 176 view of a double anti-parallel strap example embodiment used specifically for the flexed (bent) knee positioning similar to the other versions but does not provide a patella (knee cap) localizer.

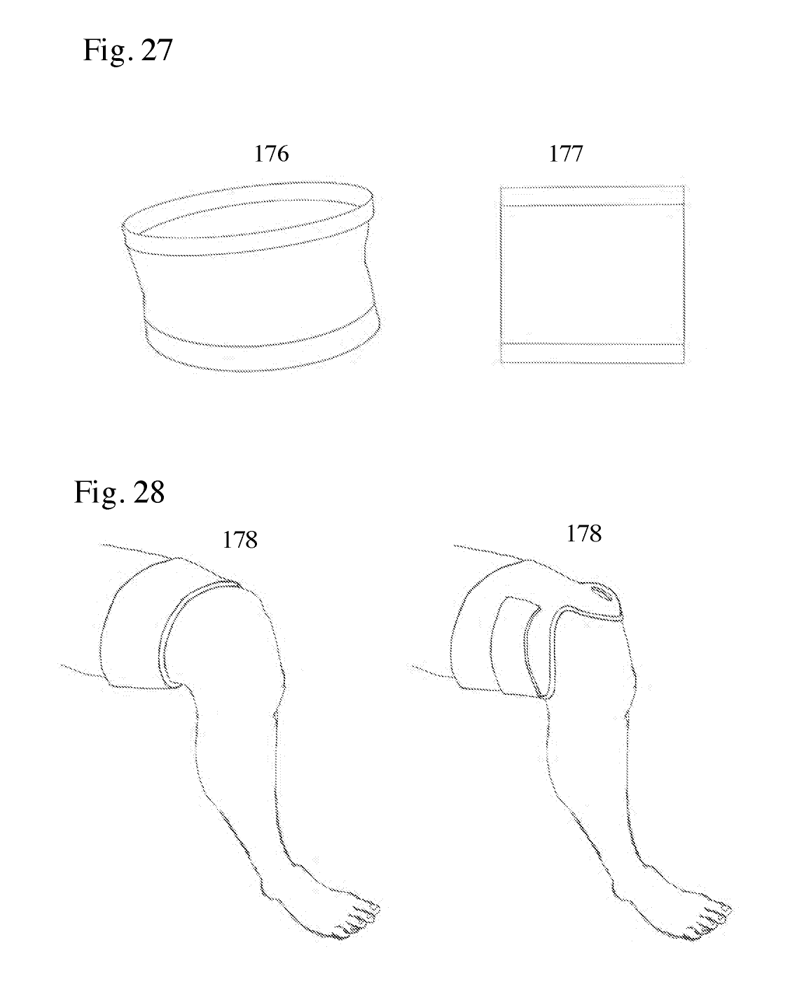

FIG. 27 is an opened 176 and folded 177 side view of a continuous ring-like example embodiment useful typically for the flexed (bent) knee positioning. The band comprises elastomeric, stretchable material that is moved up the leg to the superior knee, and compresses the suprapatellar bursa and superior knee, forcing the fluid into the inferior knee were it can be accessed.

FIG. 28 demonstrates how the suprapatellar compressive devices as in FIGS. 22-27 are placed directly above the knee (directly proximal to the knee) and then compress the suprapatellar bursa forcing fluid to the inferior knee. 178 shows the placement of the ring or band-like embodiment shown in FIG. 27 on the flexed (bent) knee. 179 shows the placement of the embodiments with patellar localized shown in FIGS. 23 and 25 on the flexed (bent) knee.

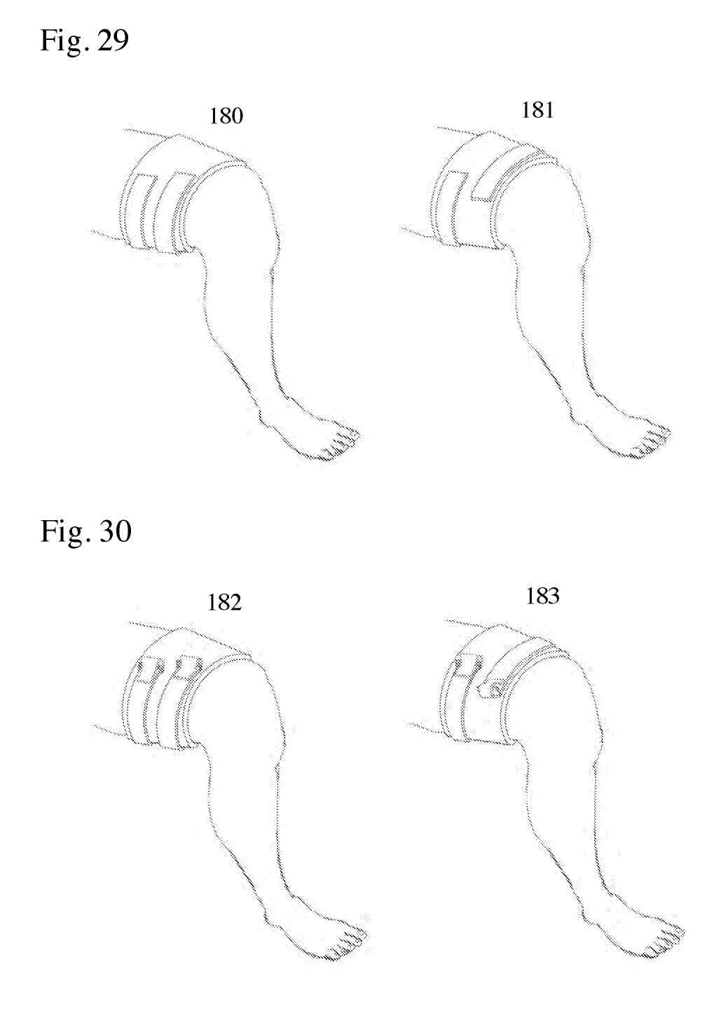

FIG. 29 demonstrates how the suprapatellar compressive devices as in FIGS. 22-27 are placed directly above the knee (directly proximal to the knee) and then compress the suprapatellar bursa forcing fluid to the inferior knee. 180 shows the placement of the parallel double strap example embodiment shown in FIG. 24 on the flexed (bent) knee. 181 shows the placement of the anti-parallel double strap example embodiment shown in FIG. 26 on the flexed (bent) knee.

FIG. 30 demonstrates the compressive devices as in FIGS. 28 and 29 with an alternative fastening mechanism, in this case a cinch mechanism with hook and pie fasteners with parallel 182 and anti-parallel 183 straps.

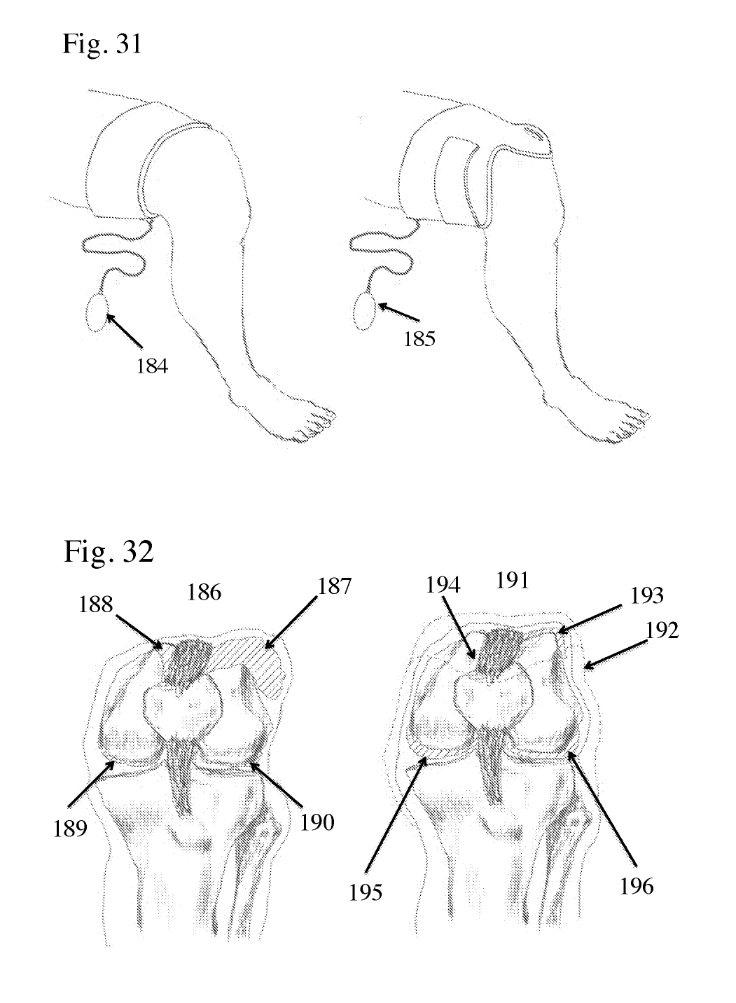

FIG. 31 shows example embodiments with air bladders that can be reversibly filled to provide desired compression. Any of the designs as in the FIGS. 22-30 can contain air bladders similar to a blood pressure cuff that are inflated with a pump device, for example the same devices used in commercial blood pressure cuffs. This embodiment can be viewed as similar to U.S. Pat. No. 7,468,048 to Meehan 2008 that has a compressive device with air bladders, but unlike the embodiments described in U.S. Pat. No. 7,468,048 to Meehan 2008 these embodiments do not have a window or aperture, are typically applied to the flexed (bent) rather than the extended knee, only encompass the superior knee (not the inferior knee), and the joint space is accessed outside the brace, not through a window or aperture as in U.S. Pat. No. 7,468,048 to Meehan 2008.

FIG. 32 shows how compressive braces as in the above embodiments that are placed on the superior knee force the fluid from the suprapatellar bursa and other knee spaces to the inferior knee, in particular the synovial reflections surrounding the medial and lateral femoral condyles and the synovial reflection of the cruciate ligaments. 186 is an anterior view of the flexed knee show fluid in the lateral suprapatellar bursa (diagonal hatch) 187 and the patellofemoral joint (diagonal hatch) 188 and much less fluid on the femoral condyles (diagonal hatch) 189 and 190, and the other inferior synovial spaces of the knee. 191 is an anterior view of the flexed knee with a compressive brace (broken line) 192 of embodiments, use, and placement as shown in FIGS. 22-31. As can be seen, the compressive brace 192 collapses the superior synovial spaces including the suprapatellar bursa (diagonal hatch) 193 and patellofemoral joint 194 and forces the fluid into the inferior knee, dilating the synovial space (diagonal hatch) over the medial 195 and lateral 196 femoral condyles and other inferior knee synovial spaces. As compared to the non-compressed knee 186, the inferior joint spaces 189 and 190, considerably dilate with fluid as in 195 and 196, and then becoming accessible to a needle.

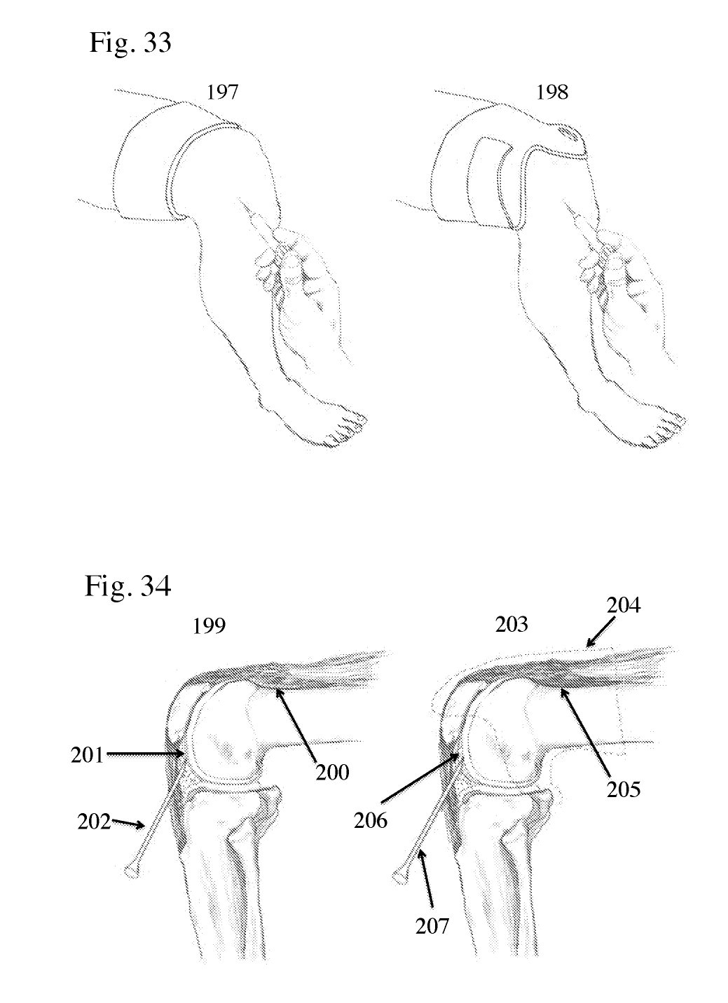

FIG. 33 shows how representative suprapatellar compressive braces 197 and 198 of the previously described designs are placed on the knee, and how the dilated synovial spaces in the inferior knee can be accessed by using the inferior portals including the lateral and medical portals defined by the inferior portion of the patella, the proximal tibia, and medial or lateral patellar tendon respectively, shown in lateral inferior portal in both 197 and 198. A design advantage of these devices over those with portals or windows previously described in this application or in in U.S. Pat. No. 7,468,048 to Meehan 2008 is that the needle is inserted outside the brace, not through a window or aperture, thus, sterility of the puncture site can be fully maintained, body fluid does not inadvertently drip on the device and thus does not contaminate it as it might with U.S. Pat. No. 7,468,048 to Meehan 2008, and the procedure can be performed in the flexed (bent) knee or sitting position. These are all of considerable practical importance that favorably affect use of the device and patient safety.

FIG. 34 shows in the non-effusive (dry)knee without a brace 199 with whatever resident fluid is present in the patellofemoral joint and suprapatellar bursa (diagonal hatch) 200, but very little in the inferior knee spaces, including the synovial spaces associated with cruciate ligaments and overlying the femoral condyles (diagonal hatch) 201. The synovial spaces in the inferior structures 201 of the flexed knee 199 are difficult and often impossible to access with a procedure needle 202 using typical inferior approaches. However, in the flexed knee 203 with a superior compressive brace (broken line) 204, the patellofemoral joint and suprapatellar bursa are collapsed 205 and whatever resident fluid is present is forced into the inferior knee, including the synovial reflections and space over the femoral condyles (diagonal hatch) dilating the space 206, where it now can be accessed by a procedure needle.

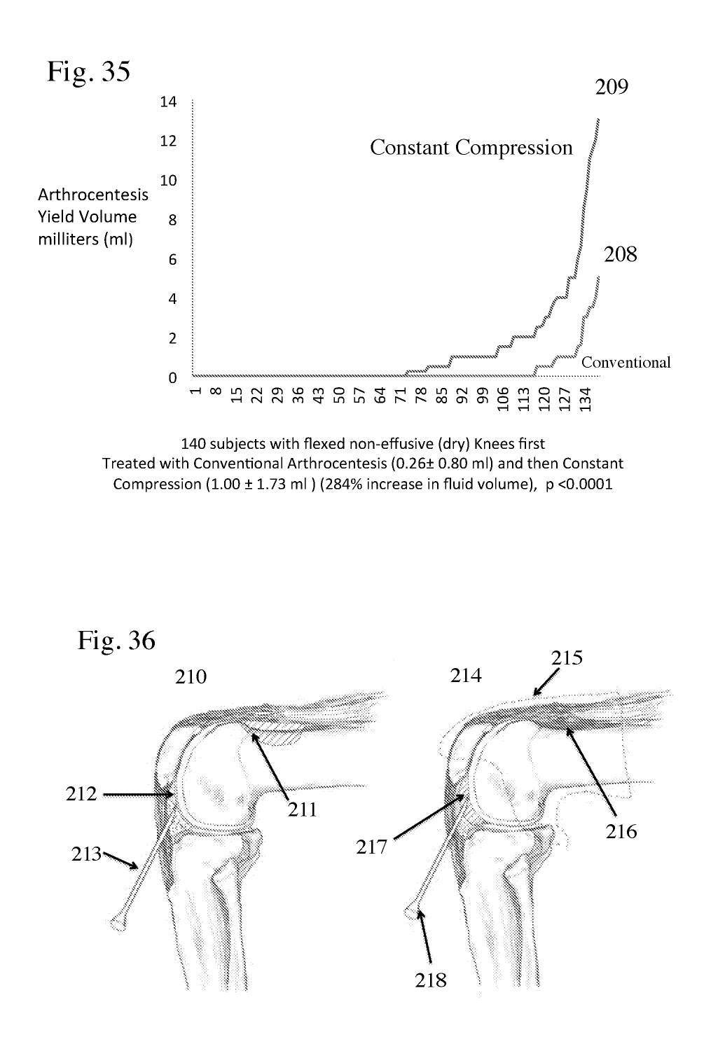

FIG. 35 is an illustration of results showing that in 140 non-effusive (dry) knees, a compressive brace as described in FIGS. 22-34 significantly increases both successful arthrocentesis (at least 2 ml of fluid) and absolute fluid yield in milliliters in the non-effusive (dry) flexed knee using the lateral inferior portal approach. The lower line 208 shows arthrocentesis yield without the compressive brace and the upper line 209 shows arthrocentesis yield with the compressive brace. As can be seen, a compressive brace according to the present invention markedly increases arthrocentesis success and yield. The compressive device increases successful arthrocentesis (at least 0.25 ml of synovial fluid) in the dry knee from 16.4% to 47.9% (p 0.000001), increases absolute diagnostic arthrocentesis (at least 2.0 ml of synovial fluid) from 4% to 22% (p 0.00001), and increases fluid yield by 284% (0.26±0.80 ml without the device to 1.00±1.73 ml with the device (p=0.0001). This improvement in needle placement and access to fluid for analysis is important and since the volumes of corticosteroid and hyaluronate injected into the joint (1 to 6 ml) are small, an aspiration of an additional 0.7 ml in the dry knee can have significant beneficial effect on drug concentration in the dry knee, increasing intraarticular drug concentrations by 10% to 45%. Further, return of synovial fluid proves the intraarticular positioning of the needle, thus, permitting more not only more complete arthrocentesis but also more accurate placement of medication by intraarticular injection.

FIG. 36 shows in the non-effusive (dry)knee without a brace 210 with whatever resident fluid is present in the patellofemoral joint and suprapatellar bursa (diagonal hatch) 211, but very little in the inferior knee spaces, including the synovial spaces associated with cruciate ligaments and overlying the femoral condyles (diagonal hatch) 212. The synovial spaces in the inferior structures 212 of the flexed knee 210 are difficult and often impossible to access with a procedure needle 213 using typical inferior approaches. However, in the flexed knee 214 with a superior compressive brace (broken line) 215, the patellofemoral joint and suprapatellar bursa are collapsed 216 and whatever resident fluid is present is forced into the inferior knee, including the synovial reflections and space over the femoral condyles (diagonal hatch) dilating the space 217, where it now can be accessed by a procedure needle 218.

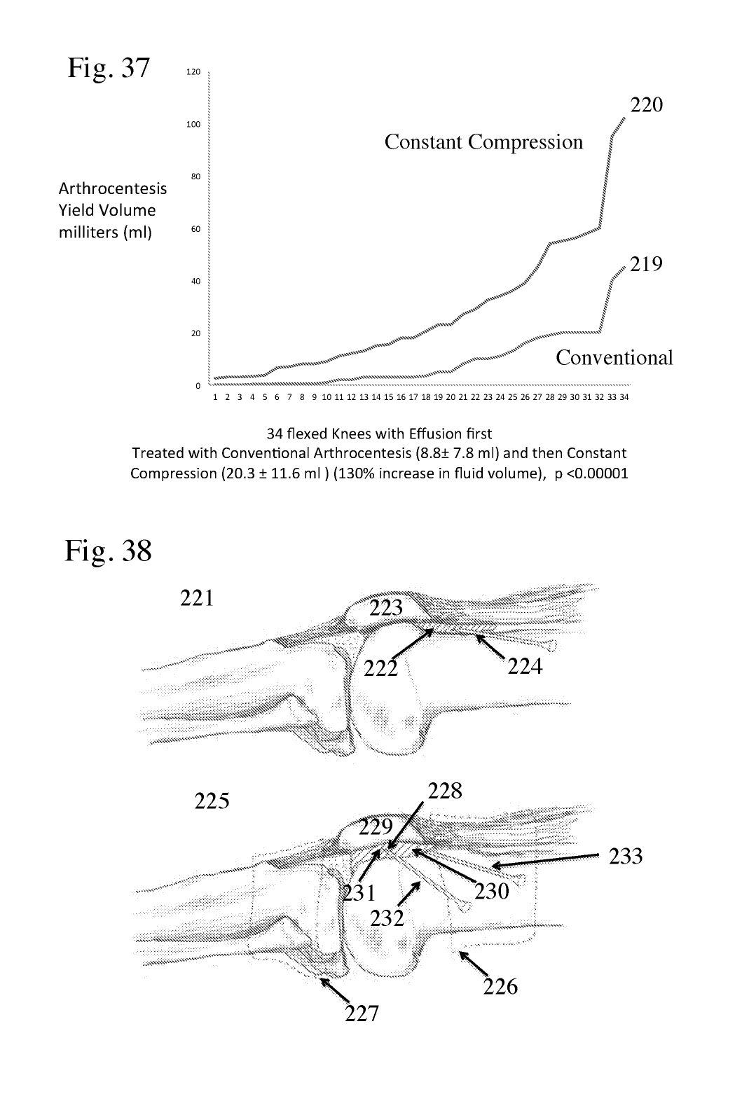

FIG. 37 is an illustration of results showing that in 34 effusive (swollen) knees in the flexed (bent) position, a compressive brace as described in FIGS. 22-34 provided significant increases in both successful arthrocentesis (at least 2 ml of fluid) and absolute fluid yield in milliliters. The lower line 219 shows arthrocentesis yield without a compressive brace and the upper line 220 shows arthrocentesis yield with the compressive brace. As can be seen, the compressive brace markedly increases arthrocentesis success and yield. The compressive device increases successful arthrocentesis (at least 0.25 ml of synovial fluid) in the dry knee from 91% to 100% (p 0.03), increases absolute diagnostic arthrocentesis (at least 2.0 ml of synovial fluid) from 79% to 100% (p 0.0007), and increases fluid yield by 130% (8.8±7.8 ml without the device to 20.3±11.6 ml with the device (p=0.00001). This improvement in needle placement and access to fluid for analysis is important and since the volumes of corticosteroid and hyaluronate injected into the joint (1 to 6 ml) are small, an aspiration of an additional 11 ml in the effusive knee may have significant beneficial effect on drug concentration in the dry knee, increasing intraarticular drug concentrations by 50% to 200%. Further, return of synovial fluid proves the intraarticular positioning of the needle, thus, permitting more not only more complete arthrocentesis but also more accurate placement of medication by intraarticular injection.

FIG. 38 shows variations of the configuration and use of the example embodiments of the compressive brace embodiments as shown in FIGS. 22 through 34. 221 is a side (lateral) view of the extended knee without the brace, 222 the synovial fluid (diagonal hatch) pools superior and posterior to the patella 223 in the patellofemoral joint and the suprapatellar bursa 224 both medially and laterally. 225 is a lateral view of the extended knee with superior compressive brace (broken line) 226 and an optional inferior compressive brace (broken line) 227 of the embodiments noted in FIGS. 22 through 35. After compressive braces 226 and optional 227 are applied to the extended effusive knee 225, the medial suprapatellar bursa collapses from the external pressure of the superior brace 226, the inferior knee synovial spaces collapses from the pressure from the inferior brace 227 and joint fluid (diagonal hatch) 228 lifts up the patella 229 dilates the proximal suprapatellar bursa 230 and patellofemoral joint 231 inferior to the patella 229. This combination dilates the targets so the patellofemoral joint 231 can be better accessed by a lateral midpatellar needle approach 232 or a lateral patellofemoral joint-suprapatellar bursa needle approach 233. A design advantage of these devices over those with portals or windows previously described in the this application or in in U.S. Pat. No. 7,468,048 to Meehan 2008 is that the needle is inserted outside the brace, not through a window or aperture, thus, sterility of the puncture site can be fully maintained even in the extended knee position, body fluid does not inadvertently drip on the device and thus does not contaminate it as it would with U.S. Pat. No. 7,468,048 to Meehan 2008, and the procedure can be performed in the extended knee position that is used most commonly to access the knee. These are all of considerable practical importance that favorably affect use of the device and patient safety.

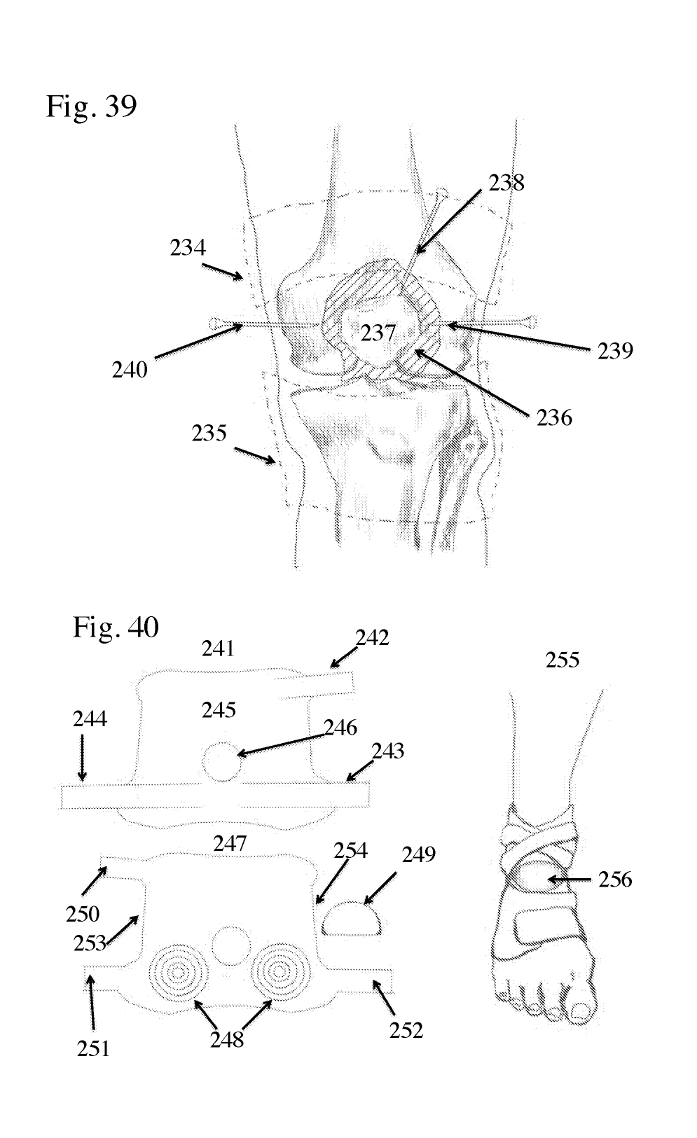

FIG. 39 shows variations of the configuration and use of the example embodiments of the compressive brace embodiments as shown in FIGS. 22 through 34 and in FIG. 38 but in an anterior extended knee view. As in FIG. 38 a superior brace (broken line) 234 and an optional inferior compressive brace (broken line) 235 apply pressure to and collapse the superior and inferior synovial fluid compartments, forcing fluid into the inferior suprapatellar bursa and primary the patellofemoral joint (diagonal hatch) 236 and lifting up the patella 237. The pooled fluid can then be accessed by a needle in the superior patellofemoral approach 238, the lateral midpatellar patellofemoral approach 239, or even the medial midpatellar patellofemoral approach 240, which is made more effective by the dilated patellofemoral joint. Use of only the superior compressive brace 235 would be effective in these approaches; applying pressure on the inferior knee with the inferior brace 235 can increase the fluid in the accessible patellofemoral joint even further. As mentioned previously, a design advantage of these devices over those with portals or windows previously described in the this application or in in U.S. Pat. No. 7,468,048 to Meehan 2008 is that the needle is inserted outside the individual braces, not through a window or aperture, thus, sterility of the puncture site can be fully maintained even in the extended knee position, body fluid does not inadvertently drip on the device and thus does not contaminate it as it would with U.S. Pat. No. 7,468,048 to Meehan 2008, and the procedure can be performed in the extended knee position that is used most commonly to access the knee. These are all of considerable practical importance that favorably affect use of the device with improved patient safety. Either or both of these devices can have pneumatic bladders as described previously.

Although the previous example embodiments are specifically useful for the knee, the invention can work on many other peripheral joints, and we provide examples for the wrist and ankle, and corresponding embodiments for other joints are anticipated and claimed.

FIG. 40 shows an embodiment with a window or aperture that is useful for the ankle, that forces fluid to the superficial ankle where it can be aspirated. 241 is a dorsal (back) view of a compressive ankle brace, with straps 242, 243, and 244, although there could be fewer or more straps as in the previous embodiments. The main panel and dorsal portion the straps 242, 243, and 244 can comprise a composite that is strong, somewhat elastomeric, and covered with pile or other fastening mechanism as previously discussed in the other embodiments. There is a cutout or opening that is largely elliptical or spherical 245 to fit the heel. 247 is a front (interior) view of the compressive ankle brace, that has two optional semi-spheroid structures 248 preferably made of closed cell foam or open foam sealed with an impermeable surface that put pressure on the pre-Achilles bursa above the heel (calcaneus) to force the fluid anteriorly. 249 is a side view of the semi-spheroid structures. The front (interior) aspect of the straps 250, 251, and 252 have hooks (Velcro) or other fasteners to attach to the dorsal side of the brace panel 245. The window or aperture to expose the ankle will be formed by the sides 253 and 254 of the panel. 255 is an anterior (front) view of the ankle with the ankle brace in place, creating a compressive brace with a low pressure window 256 over the tibiotalar joint where displaced fluid can pool. Alternative designs including a single continuous piece with a window as the embodiment in FIG. 4 are anticipated as well.

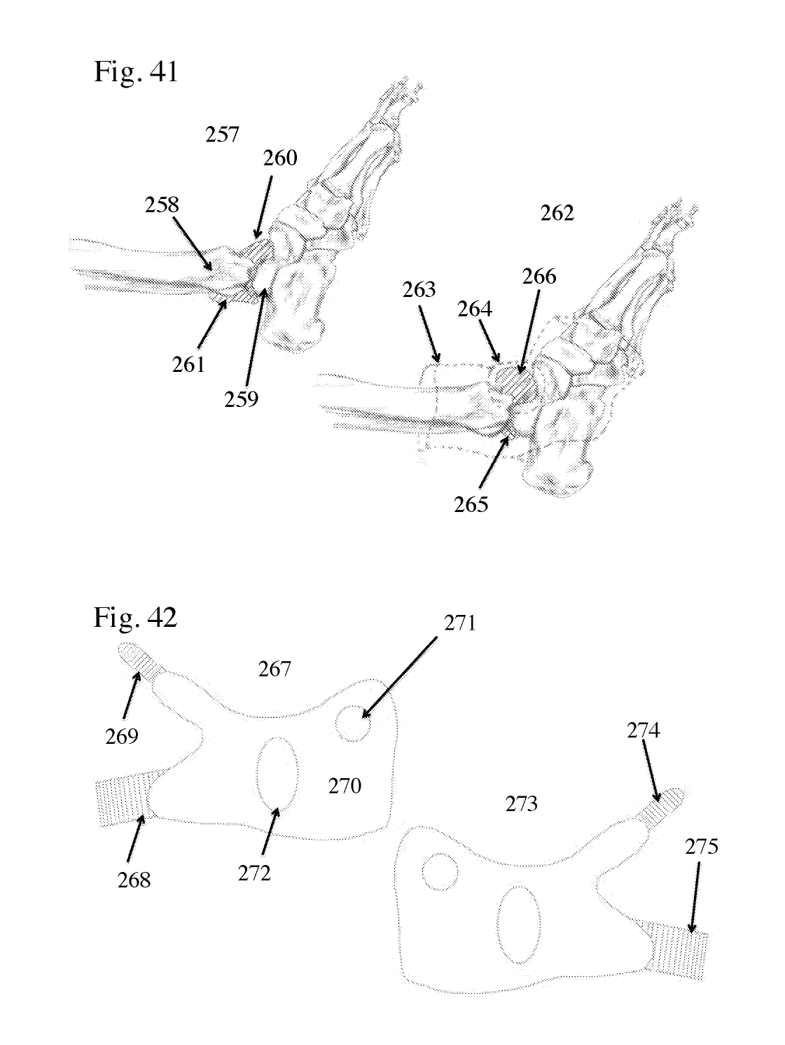

FIG. 41 shows an embodiment of a compressive ankle brace with a window that is usefu; for the ankle demonstrating the movement of fluid from the posterior ankle to the anterior ankle where it can be accessed. 257 is a medial (inner side) view of an effusive ankle without a brace. The ankle joint proper is formed by the junction of the tibia 258 and the talus 259. The fluid of the ankle pools to low-pressure areas anteriorly 260 and posteriorly 261 as well as the prepatellar bursa. 262 is a medial (inner side) view of an effusive ankle with a compressive brace (broken line) 263 with an anterior window 264. Because of pressure by the brace 263 the pre-Achilles bursa and posterior ankle effusion (diagonal hatch) 265 collapse forcing the fluid to the low pressure window 264 where an easily accessible anterior effusion (diagonal hatch) 266 pools, analogous to the previous embodiments.

FIG. 42 shows an embodiment with a window useful for the wrist. 267 is a dorsal (back) view of a compressive wrist brace, with straps 268 and 269, although there can be fewer or more straps as in the previous embodiments. The main panel 270 and dorsal portion of the straps 268 and 269 can comprise a composite that is strong, somewhat elastomeric, with portions covered with pie or other fastening mechanism as previously discussed in the other embodiments. There is a cutout or opening that is largely elliptical or spherical 271 to fit the thumb. There is also a window or aperture to permit pooling of fluid 272. 273 is a front (interior) view of the compressive wrist brace. The front (interior) aspect of the straps 274 and 275 has hooks (Velcro) or other fasteners to attach to the dorsal side of the brace panel 270. Alternative designs including a single continuous piece with a window as the embodiment in FIG. 4 are contemplated as well.

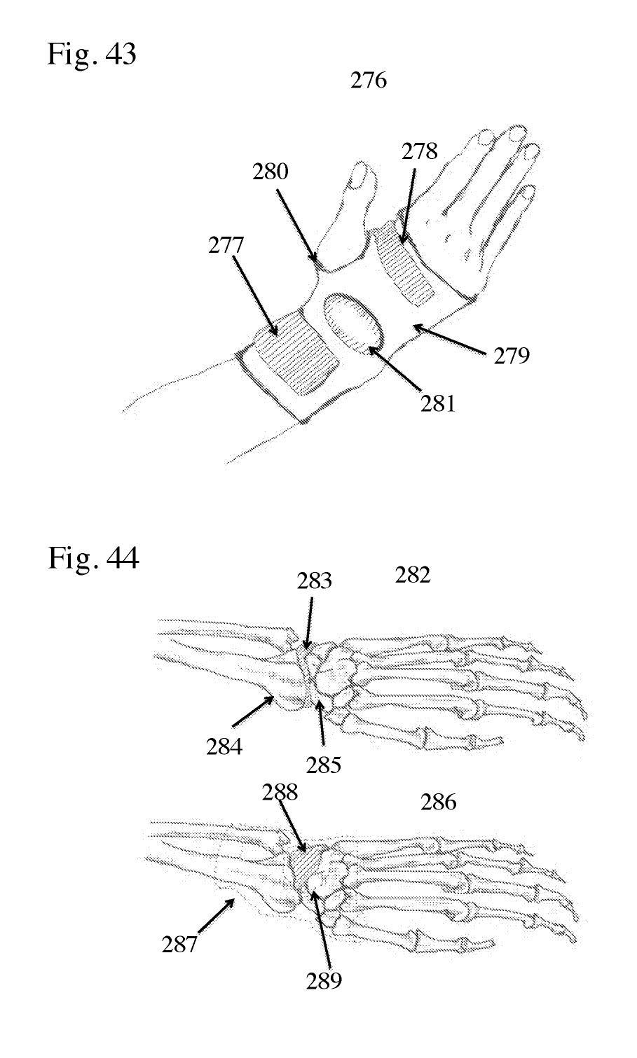

FIG. 43 shows an embodiment with a window that is useful for the wrist fastened into a functional position. 276 is a dorsal (back) view of the brace placed on the wrist. The straps with fasteners 277 and 278 bind to the panel 279 and apply pressure to the non-window sides of the wrist. The thumb protrudes from the thumb window 281. The low-pressure window 281 permits synovial fluid in the wrist to pool dorsally where it can be accessed.

FIG. 44 shows an embodiment of a compressive brace with a window that is useful for the wrist demonstrating the movement of fluid from the joint as a whole to the dorsum of the wrist where it can be accessed. 282 is a dorsal lateral (side) view of an effusive wrist without a compressive brace with the synovial structures (diagonal hatch) 283 of the wrist between and superior and inferior to the distal radius 284 and the ring of carpals 285. 286 is a dorsal lateral (side) view of an effusive wrist with a compressive wrist brace (broken line) 287 compressing the lateral, medial, and volar wrist forcing synovial fluid (diagonal hatch) 288 to pool dorsally in the low pressure area defined by the window or aperture (broken line) 289.

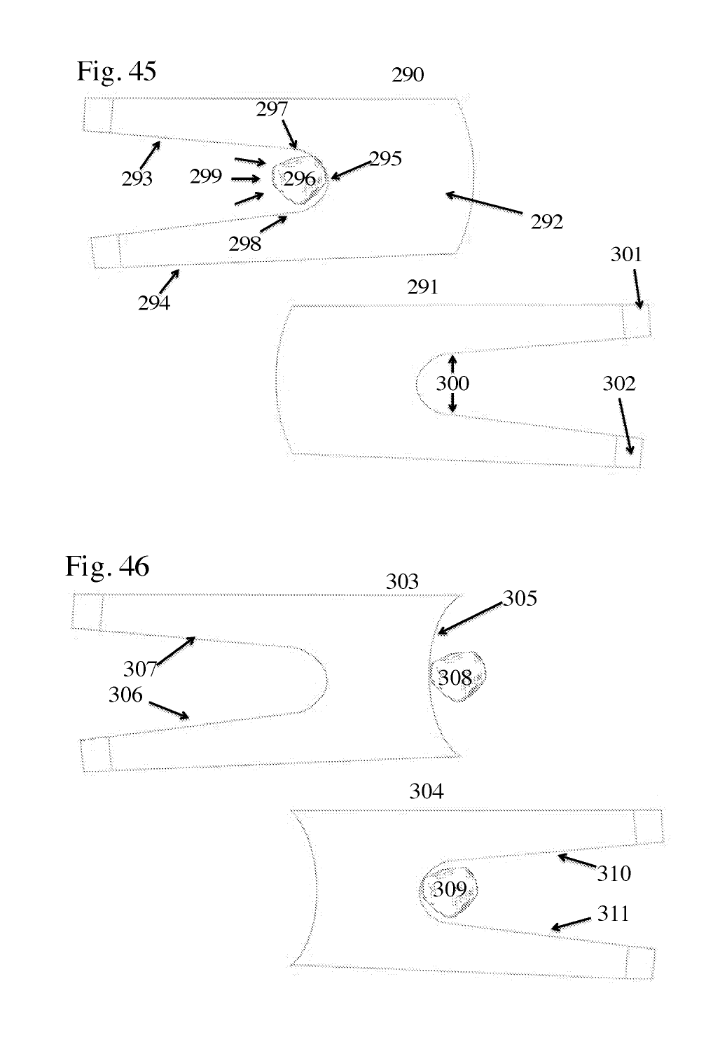

FIG. 45 is a front (anterior) 290 and back (posterior) 291 view of an alternative embodiment of the present invention where there is no window or aperture in the brace per se, rather when the brace is wrapped around the knee the low pressure window or aperture is formed by the edges of the internal straps and body. The device includes a main panel or body 292 made of a flexible (e.g., composite pie) material that is wrapped around the joint Straps 293 and 294 are used to close, tighten, and secure the device to the opposite side to provide tension to selectively apply pressure to the joint in order to cause high pressure joint fluid displacement to a low pressure targeted location at a window or aperture in this case formed by the curved junction 295 of the two straps, and the inner edges of the straps themselves 297 and 298 and cradle the patella 296 on 3 sides applying pressure to the patellofemoral joint and suprapatellar bursa and forcing fluid under the patella and to the open side 299 of the patella 296 where the patella can be accessed (three arrows). The distance 300 between the straps at the point of patellar accommodation needs to be large enough to accommodate typical patella superior-inferior dimensions, typically 4-9 cm. The straps 293 and 295 include securing elements, such as a male/female interdigitating structures for example hooks or other fasteners 301 and 302 that attach to corresponding pile material (such as Velcro fasteners) or other fasteners located on the backside of the main panel 292. It is contemplated that a pneumatic version of this brace without an intrinsic window or aperture with reversibly inflatable internal air bladders will also function in an identical fashion.

FIG. 46 is a front (anterior) 303 and back (posterior) 304 view of an example embodiment of the present invention where there is no window or aperture in the brace per se similar to the embodiment in FIG. 45, but the window or aperture is formed by the far end of the body 305 and the more distal internal edges of the straps 306 and 307, that closely surround the patella 308 on three sides. It is contemplated there could be a third strap as well. The far edge of the body 305 can be curved as shown, although other configurations are possible. 304 demonstrates that in this embodiment if configured in dimension the same as in 303 the patella 309 can also be accommodated between the straps 310 and 311 if the brace is positioned as in FIG. 45. Thus, there is an option in this embodiment to position the brace relative to the patella in these two different configurations for the same purpose. It is anticipated that a pneumatic version of this brace without an intrinsic window or aperture with reversibly inflatable internal air bladders would also function in an identical fashion.

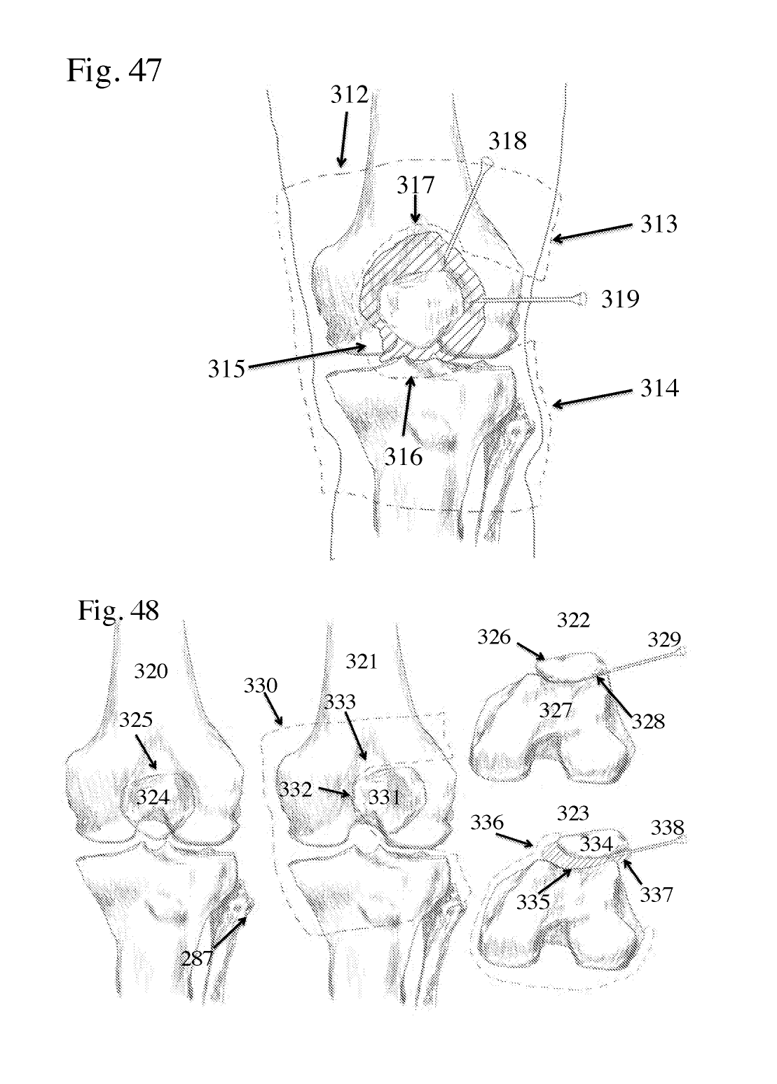

FIG. 47 is a front (anterior) view of the knee with a compressive brace 312 (broken line) of the embodiments of FIG. 45 and FIG. 46 in place with the straps 313 and 315 fastened forming a 3 sided compression with one side being the body panel of the brace 315 (broken line) and the two straps 316 and 317 (broken line). With pressure provided by the body panel 315 over the opposing side of the knee, the superior strap 317 over the suprapatellar bursa and superior patellofemoral joint, and the inferior strap 316 applying pressure over the inferior patellofemoral joint and femoral tibial joint, synovial fluid (diagonal hatch) is forced into the lateral and central patellofemoral joint where it can be accessed by traditional needle approaches 318 and 319. The brace can be rotated 180 degrees and the opposite side of the knee can be accessed in a identical manner. As the figure shows, the concentration of fluid in the patellofemoral joint is such that traditional approaches to arthrocentesis and injection can be utilized. The aperture in this position of the knee only requires three sides (315, 316, and 317) because the suprapatellar bursa is limited in the extent of posterior displacement and expansion by natural anatomic limitations of the synovial structures laterally in the midknee.

FIG. 48 demonstrates how the constant compressive brace of embodiments as in FIGS. 45-47 can displace the patella to permit a needle to be more easily passed into the knee as well as providing greater success in aspirating fluid. 320 is anterior view of a knee without a compressive brace, 321 is an anterior view with a compressive brace, 322 is an axial view of the knee without a compressive brace, and 323 is an axial view with a constant compressive brace in place. In the anterior view 320 the patella 324 rides in the patella femoral joint 325 in the midline; in the axial view 322, the patella 326 rides on the femur 327, and the patellofemoral joint 328 is very tight and difficult to pass a needle 329 into the very tight patellofemoral joint 328. When the brace 330 (broken line) is applied to knee, the patella 331 is displaced laterally by the leading edge 332 of the brace forcing the patella 331 to the lateral side of the knee, while the brace forces fluid under the patella and into the lateral patellofemoral joint 333 is the axial view of the knee, showing the patella 334 displaced laterally by the compressive brace 336 (broken line) to the femoral side of the patellofemoral joint, and dilating the patellofemoral joint with fluid (diagonal hatch). Since the patella 334 is displaced laterally and the patella lifted by fluid (diagonal hatch) the lateral patellofemoral joint 337 is much larger and can be more easily accessed by the procedure needle 338.

FIG. 49 is an illustration of results that show that, when used in 430 unselected subjects with symptomatic osteoarthritis of the knee undergoing arthrocentesis and then injection with 60-80 mg triamcinolone acetonide; 215 with the constant compression brace and 215 with conventional arthrocentesis and injection, the constant compression group has a longer time to next injection (until next symptomatic joint flare) thus increasing the effectiveness of the injected medication and increasing the cost-effectiveness of injection procedure: Constant compression 6.9±3.5 months, Conventional 5.1±2.7 months (35% increase), 95% Cl of difference: 1.2091<1.8<2.3909, p=0.00001.

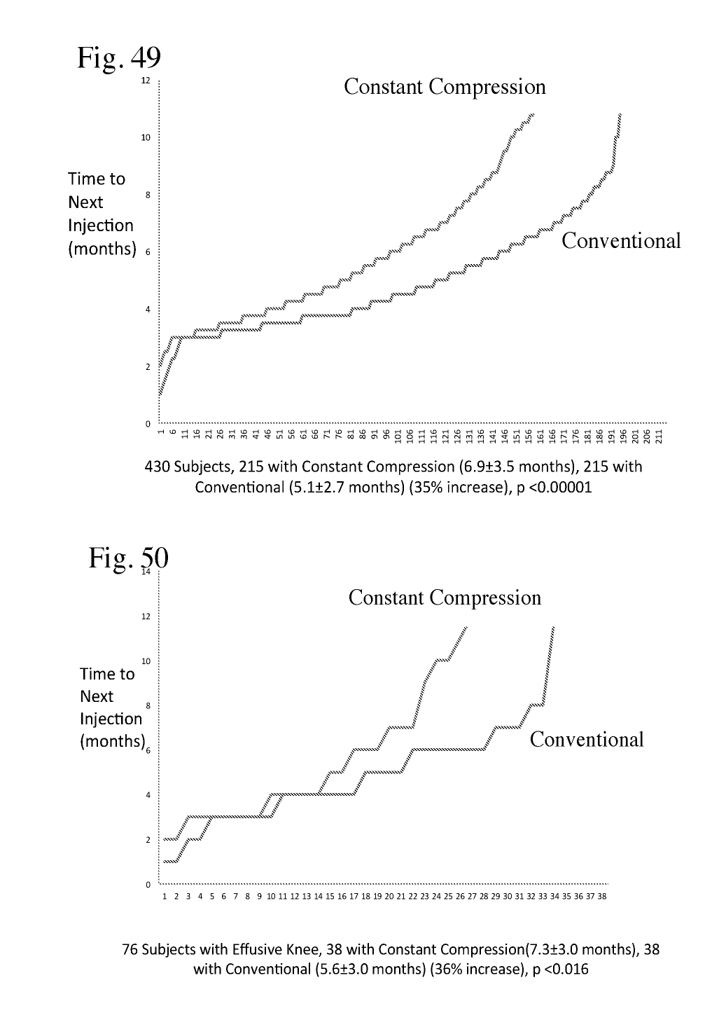

From the data of Weitoft 2000, it would be expected that more complete arthrocentesis in the effusive knee, would be expected to improve the outcome injections in those patients. FIG. 50 shows that, when used in 76 subjects with clinically effusive (swollen) knee, 38 with the constant compression brace and 38 without constant compression, the constant compression group has a longer time to next injection (until next symptomatic joint flare) thus increasing the effectiveness of the injected medication and increasing the cost-effectiveness of injection procedure: Constant compression 7.3±3.0 months, Conventional 5.6±3.0 months (36% increase), 95% Cl of difference: −3.058<−1.7<−0.342, p=0.016. This indicates that more complete arthrocentesis in the effusive permitted by the constant compression brace does significantly improve the outcome of injection procedures in the effusive knee.

However, in the non-effusive (dry knee), it would be difficult to understand why a compressive brace might also improve the outcome, excepting that the compressive brace does still provide unexpected fluid in the non-effusing knee, as shown in FIG. 35 and increases access to synovial compartments as shown in FIG. 48. To address this question, we examined subjects with non-effusive (dry) knee.

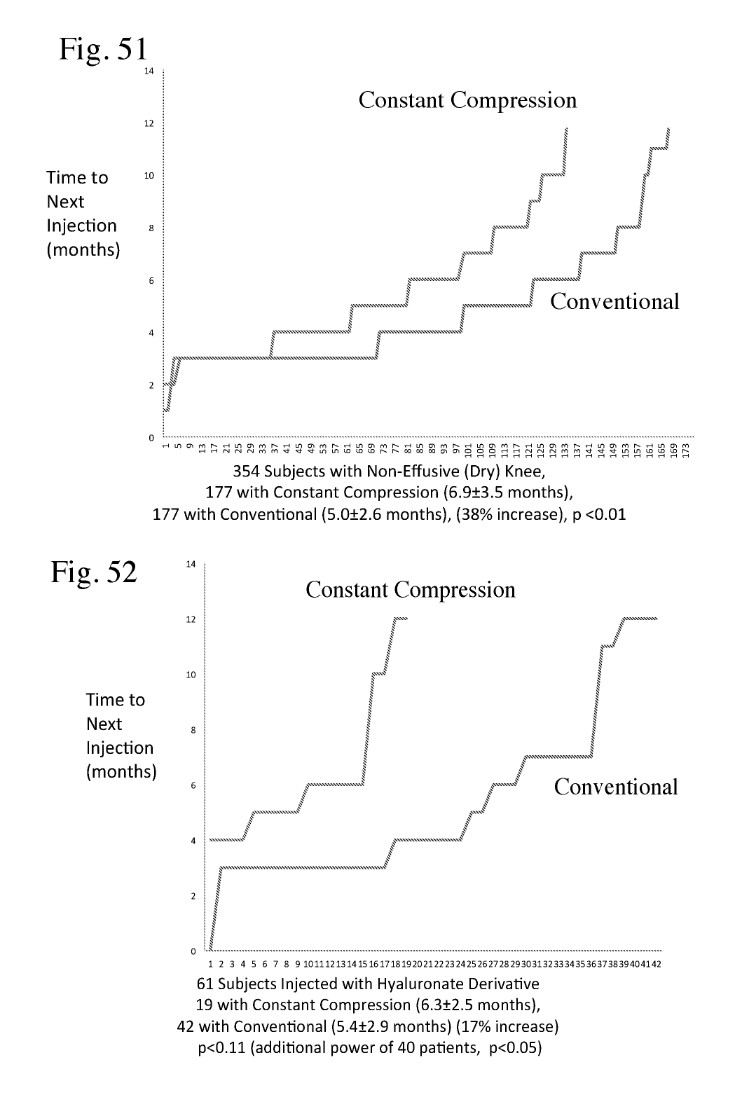

FIG. 51 shows that, when used in 354 subjects with clinically non-effusive (dry) knee, 177 with the constant compression brace and 177 without constant compression, the constant compression group has a longer time to next injection (until next symptomatic joint flare) thus increasing the effectiveness of the injected medication and increasing the cost-effectiveness of injection procedure: Constant compression 6.9±3.5 months, Conventional 5.0±2.6 months (38% increase), 95% Cl of difference: −3.2983<−1.9<−0.5017, p=0.01. This data show that the constant compression brace in the clinically non-effusive knee also does significantly improve the outcome of injection procedures, and unexpected outcome, but very useful clinically.

The above data demonstrating enhance clinical response were obtained with corticosteroid, but our studies indicate that the same beneficial effect defined by enhanced response and duration of action caused by the use of a compressive brace also occurs with intraarticular injection of hyaluronate derivatives, and we also claim the combination of a compressive brace and intraarticularly injected hyaluronate derivatives.

FIG. 52 shows results that show that, when used in 61 subjects with osteoarthritis of the knee, 19 with the constant compression brace and 42 without constant compression, the constant compression group has a longer time to next injection (until next symptomatic joint flare) thus increasing the effectiveness of the injected medication and increasing the cost-effectiveness of injection procedure. Constant compression 6.3±2.5 months, Conventional 5.4±2.9 months (17% increase), p=0.11 which is a strong trend, with a power calculation if 40 more patients were studied, the p value would be <0.05. Thus, the same effect with hyaluronates is noted.

It is contemplated that this beneficial effect will also be present with the combination of a compressive brace and intraarticularly injected biologic response modifiers, peptides, proteins, bacterial and viral products, genetic material (DNA, RNA, derivatives), and any other intraarticular therapy or device.

These devices can be especially useful in (1) diagnostic arthrocentesis, (2) therapeutic arthrocentesis, (3) joint therapy where medications are injected into the joint, (4) traumatic arthritis and infectious arthritis to fully remove blood and pus, (5) harvesting of synovial and aspirate fluid for cells and biomarkers, and (6) diagnostic imaging and orthopedic surgery.

Example embodiments above are shown to be reduced to practice with beneficial effect on diagnostic and therapeutic arthrocentesis with more accurate needle placement. In intraarticular joint therapy, the device permits the needle to be more accurately placed in the joint space (as proven by return of synovial fluid), which improves the accuracy and outcome of the injection, and permits full decompression of the joint that causes the injected drugs to be more concentrated and thus more effective as shown in FIGS. 49-51. In traumatic and infectious arthritis the device permits more complete aspiration of harmful synovial fluid contents.

A device as described herein is also useful for injecting medication into the joint (intraarticular injections), including corticosteroid, hyaluronate, saline, cells, drugs, or any other substance or medication injected into the joint and provides a better outcome as shown in FIGS. 49-51.

A device as described herein is useful to permit or improve the process of intraarticular needle placement, arthrocentesis, or complete joint decompression to facilitate intraarticular injection and improve the outcome of medications injected into the joint including corticosteroids, hyaluronate, and other joint therapies.

A device as described herein is useful singly or as part of a system to harvest joint fluid, intraarticular blood, or cells for biomarkers, cultures, genetic analysis and other diagnostic and monitoring purposes.

A device as described herein is useful also useful for needle or instrument placement in orthopedic surgery and diagnostic imaging and image-guided procedures, including ultrasound, magnetic resonance imaging, standard radiography, computed tomography, contrast enhanced procedures, and fluoroscopy.

A device as described herein can also immobilize a joint during the procedure and decrease patient motion. A device as described herein, by providing pressure, distraction, pressure on sensory nerves, counter irritation, and neurologic stimulation, can decrease the pain of joint procedures performed while the device is affixed.

It is also contemplated that with appropriate size modifications the present invention will provide similar benefits to joints other than the knee, and provide embodiments for the wrist and ankle have been described, thus, we anticipate similar devices with similar function for other joints.

While the present invention has been disclosed above with respect to various preferred embodiments, it shall be understood that changes and modifications may be made to the invention in accordance with the scope of the claims appended hereto.

### CITATION LIST

### Patent Literature

U.S. Pat. No. 4,489,718 December 1984 Martin
U.S. Pat. No. 4,872,448 October 1989 Johnson, Jr.
U.S. Pat. No. 4,953,543 September 1990 Grim et al.
U.S. Pat. No. 4,960,115 October 1990 Ranciato
U.S. Pat. No. 5,038,938 August 1991 Berndt
U.S. Pat. No. 5,107,823 April 1992 Fratesi
U.S. Pat. No. 5,139,477 August 1992 Peters
U.S. Pat. No. 5,334,135 August 1994 Grim et al.
U.S. Pat. No. 5,451,201 September 1995 Prengler
U.S. Pat. No. 563,490 June 1997 Battenfield
U.S. Pat. No. 570,137 December 1997 Muks et al.
U.S. Pat. No. 578,567 July 1998 Billotti
U.S. Pat. No. 579,208 August 1998 Wilson et al.
U.S. Pat. No. 580,264 September 1998 Ferrand et al.
U.S. Pat. No. 582,398 October 1998 Grim et al.
U.S. Pat. No. 584,606 December 1998 Lakic
U.S. Pat. No. 585,799 January 1999 Thomas et al.
U.S. Pat. No. 586,516 February 1999 Fitzpatrick et al.
U.S. Pat. No. 590,601 May 1999 Ferrand et al.
U.S. Pat. No. 590,601 May 1999 Ferrand et al.
U.S. Pat. No. 592,505 July 1999 Thomas et al.
U.S. Pat. No. 595,787 September 1999 Klein
U.S. Pat. No. 601,047 January 2000 Wycoki
U.S. Pat. No. 604,260 March 2000 Wells
U.S. Pat. No. 612,759 October 2000 Beyar et al.
U.S. Pat. No. 614,961 November 2000 Klein
U.S. Pat. No. 620,028 March 2001 Zamani
U.S. Pat. No. 625,337 July 2001 Ritter
U.S. Pat. No. 625,678 July 2001 Tyler
U.S. Pat. No. 631,239 November 2001 Cencer
U.S. Pat. No. 637,440 April 2002 Tomlinson et al.
U.S. Pat. No. 643,877 August 2002 Ferrand et al.
U.S. Pat. No. 649,767 December 2002 Rogalski
U.S. Pat. No. 652,773 March 2003 Ceriani
U.S. Pat. No. 6,527,760 March 2003 Vad
U.S. Pat. No. 655,128 April 2003 Knighton et al.
U.S. Pat. No. 657,257 June 2003 Klein
U.S. Pat. No. 658,577 July 2003 Dean, Jr. et al.
U.S. Pat. No. 659,253 July 2003 Hotchkiss et al.
U.S. Pat. No. 666,966 December 2003 Branch
U.S. Pat. No. 676,172 July 2004 Findlay et d.
U.S. Pat. No. 677,920 August 2004 Shah
U.S. Pat. No. 678,264 August 2004 Westin
U.S. Pat. No. 684,820 February 2005 Westin
U.S. Pat. No. 686,671 March 2005 Shutic
U.S. Pat. No. 693,950 September 2005 Lyden
U.S. Pat. No. 694,159 September 2005 Ferrand
U.S. Pat. No. 701,835 March 2006 Iglesias
U.S. Pat. No. 704,164 May 2006 Rueger
U.S. Pat. No. 705,617 June 2006 Courtney
2001/001466 August 2001 Rueger
2001/001664 August 2001 Rueger
2002/014337 October 2002 Courtnage et al.
2003/019143 October 2003 Knighton
2003/022642 December 2003 Gotlib
2003/022932 December 2003 Simon
2004/022049 November 2004 Findlay
2005/004366 February 2005 Stark et al.
2005/009662 May 2005 Howard
2005/010186 May 2005 Ridley
2005/022138 October 2005 Liew
2005/028312 December 2005 Simon
2006/017342 August 2006 Urich
2006/017747 August 2006 Rueger
U.S. Pat. No. 7,468,048 December 2008 Meehan
D746,997 Jan. 5, 2016 Higgins

### NonPatent Literature

Chavez-Chiang C E, Sibbitt W L Jr, Band P A, Chavez-Chiang N R, Delea S L, Bankhurst A D. The highly accurate anterioateral portal for injecting the knee. Sports Med Arthrosc Rehabil Ther Technol. 2011 Mar. 30; 3(1):6. doi: 10.1186/1758-2555-3-6.PMID: 21447197
Lawrence D, Kakkar W. Graduated, static, external compression of the lower limb: a physiological assessment. Br J Surg. 1980 February; 67(2):119-21.
Janssen H, Treviño C, Williams D. Hemodynamic alterations in venous blood flow produced by external pneumatic compression. J Cardiovasc Surg (Torino). 1993 October; 34(5):441-7.
Jackson D W, Evans N A, Thomas B M. J Accuracy of needle placement into the intra-articular space of the knee. Bone Joint Surg Am. 2002; 84-A(9):1522-7.
Westrich G H, Specht L M, Sharrock N E, Windsor R E, Sculco T P, Haas S B, Trombley J F, Peterson M. Venous haemodynamics after total knee arthroplasty: evaluation of active dorsal to plantar flexion and several mechanical compression devices. J Bone Joint Surg Br. 1998 November; 80(6):1057-66.
Kuster M S, Grob K, Kuster M, Wood G A, Gächter A. The benefits of wearing a compression sleeve after ACL reconstruction. Med Sci Sports Exerc. 1999 March; 31(3):368-71.
Ike R W, Arnold W J, Rothschild E W, Shaw H L Tidal irrigation versus conservative medical management in patients with osteoarthritis of the knee: a prospective randomized study. Tidal Irrigation Cooperating Group. J Rheumatol. 1992 May; 19(5):772-9.
Bradley J D, Heilman D K, Katz B P, Gsell P, Wallick J E, Brandt K D.
Tidal irrigation as treatment for knee osteoarthritis: a sham-controlled, randomized, double-blinded evaluation. Arthritis Rheum. 2002 January; 46(1):100-8.
Arden N K, Reading I C, Jordan K M, Thomas L, Platten H, Hassan A, Ledingham J. A randomised controlled trial of tidal irrigation vs corticosteroid injection in knee osteoarthritis: the KIVIS Study. Osteoarthritis Cartilage. 2008 June; 16(6):733-9. Epub 2008 Feb. 20.
Nahir A M, Schapira D, Besser M, Scharf Y. Vacuum arthrocentesis for massive knee effusions. Br J Rheumatol. 1984 August; 23(3):235-6.
Roberts W N, Hayes C W, Breitbach S A, Owen D S Jr. Dry taps and what to do about them: a pictorial essay on failed arthrocentesis of the knee. Am J Med. 1996 April; 100(4):461-4.
Weitoft T, Uddenfeldt P. Importance of synovial fluid aspiration when injecting intra-articular corticosteroids. Ann Rheum Dis. 2000 March; 59(3):233-5.
Tanaka N1, Sakahashi H, Sato E, Hirose K, Ishima T, Ishii S. Intra-articular injection of high molecular weight hyaluronan after arthrocentesis as treatment for rheumatoid knees with joint effusion. Rheumatol Int. 2002 August; 22(4):151-4. Epub 2002 Jun. 26.
Zuber T J. Knee joint aspiration and injection. Am Fam Physician. 2002 Oct. 15; 66(8):1497-500, 1503-4, 1507.
Wiler J L1, Costantino T G, Flippone L, Satz W. Comparison of ultrasound-guided and standard landmark techniques for knee arthrocentesis. J Emerg Med. 2010 July; 39(1):76-82. doi: 10.1016/j.jemermed.2008.05.012. Epub 2008 Dec. 5.
Ike R W, Somers E C, Arnold E L, Arnold W J. Ultrasound of the knee during voluntary quadriceps contraction: a technique for detecting otherwise occult effusions. Arthritis Care Res (Hoboken). 2010 May; 62(5):725-9. doi: 10.1002/acr.20047. PMID: 20461790
Sibbitt W L Jr, Kettwich L G, Band P A, Chavez-Chiang N R, DeLea S L, Haseler U, Bankhurst A D. Does ultrasound guidance improve the outcomes of arthrocentesis and corticosteroid injection of the knee? Scand J Rheumatol. 2012 February; 41(1):66-72. doi: 0.3109/03009742.2011.599071. Epub 2011 Nov. 21.PMID: 22103390
Hirsch G, O'Neill T, Kitas G, Klocke R. Distribution of effusion in knee arthritis as measured by high-resolution ultrasound. Clin Rheumatol. 2012 August; 31(8):1243-6. doi: 10.1007/s10067-012-1987-3. Epub 2012 Apr. 24
Zhang Q, Zhang T, Lv H, Xie L, Wu W, Wu J, Wu X. Comparison of two positions of knee arthrocentesis: how to obtain complete drainage. Am J Phys Med Rehabil. 2012 July; 91(7):611-5. doi: 10.1097/PHM.0b013e31825a13f0. PMID: 22710881
Wu T, Dong Y, Song H X, Fu Y, Li J H. Ultrasound-guided versus landmark in knee arthrocentesis: A systematic review.
Semin Arthritis Rheum. 2015 Dec. 17. pii: S0049-0172(15)00272-3. doi: 10.1016/j.semarthrit2015.10.011. [Epub ahead of print]
Zhang Q, Zhang T. Effect on Pain and Symptoms of Aspiration Before Hyaluronan Injection for Knee Osteoarthritis: A Prospective, Randomized, Single-blind Study. Am J Phys Med Rehabil. 2015 Oct. 20. [Epub ahead of print]
Bhavsar T, Sibbitt W Jr., Cabacungan R, Moore T, Salayandia L, Fields R, Bankhurst A, Emil S, Fangtham M, Konstantinov K.: Improvement in Knee Arthrocentesis Via Constant Compression. Arthritis Rheumatol Page: 68, 11 2016 Notes: http://acrabstracts.org/abstract/improvement-in-knee-arthrocentesis-via-constant-compression/

1. A non-pneumatic compression device comprising an open planar structure made of cloth, plastic, elastic, elastomeric, composite, or other material, configured such that when wrapped around a joint the sleeve provides circumferential pressure to the soft tissues surrounding the joint thereby urging synovial fluid to desired low pressure areas of the joint from where it can be removed by arthrocentesis, wherein the sleeve further comprises fastening elements configured to affix the opposing ends of the structure and secure the sleeve around the joint.

2-42. (canceled)

43. A method of introducing a needle into a desired region of a joint, comprising:
(a) providing a compression device comprising an open planar structure made of cloth, plastic, elastic, elastomeric, composite, or other material, configured such that when wrapped around the joint the sleeve provides circumferential pressure to soft tissue surrounding the joint which pressure urges synovial fluid toward a desired accessible region of the joint and away from an inaccessible region of the joint;
(b) placing the compression device on the joint such that an elevated synovial fluid volume distends the synovial membrane in the desired region;
(c) introducing the needle into the desired region.

44. A method as in claim 43, wherein the structure defines a first access portal, and wherein step (b) comprises placing the compression device on the joint such that the desired region is accessible via the first access portal.

45. A method as in claim 44 wherein the joint is a knee joint, and wherein step (b) comprises placing the compression device such that one or more of: the medial or lateral suprapatellar bursa, the lateral recess of the suprapatellar bursa, the medial or lateral patellofemoral joint, the suprapatellar bursa and synovial reflections dorsal to (under) the quadriceps tendon, and the synovial reflections overlying the lateral and medial femoral condyles and cruciate ligaments are accessible via the first access portal.

46. A method as in claim 43, wherein the sleeve further comprises one or more straps fixed or fixable to the structure at first and second ends of the straps, and wherein step (b) comprises placing the compression device on the joint such that there is a space between straps or between a strap and the structure, and the desired region is accessible via the space.

47. A method as in claim 43, wherein the joint is a knee joint, and wherein the structure comprises a strap from 1 cm to 25 cm in width and 10 cm to 100 cm in length, and wherein step (b) comprises placing the strap about the knee including or starting at or proximal to the patella, including compression to the suprapatellar bursa so that intraarticular fluid is urged inferiorly, so that medial or lateral portals inferior to the patella (knee cap) can be accessed by the needle.

48. A method as in claim 43, wherein the device further comprises internal bladders configured to reversibly apply pneumatic pressure to regions of the joint.

49. A method as in claim 43, wherein the device can be adjusted about the joint to provide variable pressure, and wherein step (b) comprises adjusting the device to provide a desired internal pressure.

50. A method as in claim 43, wherein the structure is 5 cm to 45 cm in width and 10 cm to 100 cm in length.

51. A method as in claim 43, further comprising aspirating fluid from the joint via the needle after step (c).

52. A method as in claim 43, further comprising injecting medication into the joint via the needle after step (c).

53. A method as in claim 52, wherein the medication comprises one or more of corticosteroid, hyaluronate derivatives, saline, cells, drugs, and biological response modifiers.

54. A method as in claim 43, further comprising, after step (c), subjecting the joint to one or more of ultrasound, magnetic resonance imaging, standard radiography, computed tomography, contrast enhanced procedures, and fluoroscopy.

55. A method as in claim 44, wherein the device comprises a marker that facilitates placement of the device and the first access portal in a reproducible anatomic position in relation to underlying palpable or visible anatomy.

56. A method as in claim 55, wherein the marker comprises one or more of: a surface marker on the device, a cutout on the device, or extension or structure of the first access portal.

57. A method as in claim 44, wherein the device comprises a first access portal cover, configured to reversibly cover the first access portal.

58. A method as in claim 44, wherein the device defines a second access portal, and wherein step (c) comprises placing an ultrasound probe in communication with the joint via the second access portal, and placing the needle responsive to signals from the ultrasound probe.

59. The method of claim 43, wherein the joint is a knee joint, and wherein the device is configured to physically displace the patella to the medial or lateral side, and wherein step (c) comprises introducing the needle into the patellofemoral joint or the suprapatellar bursa.

60. The method of claim 43, wherein the joint is a knee joint, and wherein the device is configured to displace fluid under the patella and expand the patellofemoral joint and suprapatellar bursa, and wherein step (c) comprises introducing the needle into the patellofemoral joint or the suprapatellar bursa.

61. The method of claim 43, wherein the joint is a knee.

62. The method of claim 44, wherein the access portal defines an opening that is at least 4 cm across in at least one dimension.
